(19) United States
(12) Patent Application Publication
(10) Pub. No.: US 2023/0307092 A1
(43) Pub. Date: Sep. 28, 2023

(54) IDENTIFYING GENOME FEATURES IN HEALTH AND DISEASE

(71) Applicant: GENOME INTERNATIONAL CORPORATION, Madison, WI (US)

(72) Inventors: Periannan Senapathy, Madison, WI (US); Sudar Senapathy, Madison, WI (US)

(21) Appl. No.: 18/188,389

(22) Filed: Mar. 22, 2023

Related U.S. Application Data
(60) Provisional application No. 63/323,287, filed on Mar. 24, 2022.
(60) Provisional application No. 63/355,957, filed on Jun. 27, 2022.

(51) Int. Cl. G16B 20/20; G16B 40/20; G16B 30/00; G06N 20/00

(52) U.S. Cl. CPC G16B 20/20; G16B 40/20; G16B 30/00; G06N 20/00

(57) ABSTRACT

Presented herein are methods and systems directed to analysis of features, mutations, and genome sequences. Analysis of genetic features can identify strongly or weakly causative deleterious mutations.

### INCORPORATION BY REFERENCE TO ANY PRIORITY APPLICATIONS

This application claims priority to U.S. Provisional App. No. 63/323,287, filed Mar. 24, 2022, and to U.S. Provisional App. No. 63/355,957, Filed Jun. 27, 2022. Any and all applications for which a foreign or domestic priority claim is identified in the Application Data Sheet as filed with the present application are hereby incorporated by reference under 37 CFR 1.57.

### BACKGROUND OF THE INVENTION

The ability to sequence a genome faster and cheaper using novel Next Generation Sequencing (NGS) technology is revolutionizing the field of Precision Medicine. This field is expected to improve the diagnosis and treatment of numerous diseases based on the genome sequence of an individual. In addition, basic and clinical research in this field have been expanding due to the rapid advancements of NGS technologies.

Research in any field progresses based on the findings that have been described in previously published research articles. The research community relies on the easy availability of these publications in user friendly platforms. There are several public resources, such as PUBMED and Google Scholar, as well as commercial resources that collect and enable the search and retrieval of biomedical and life sciences literature. Most of these sources allow the searching of publications based on defined search terms and the years of publications, and provide the results as a set of publications that fulfill these criteria. Each of these resources provide several functionalities for searching and viewing the results.

In addition to these capabilities, the field of Precision Medicine will benefit from the ability to analyze the results further in advanced ways, running instant meta-analysis using extant results. Such work requires searching beyond the general capabilities of data retrieval and viewing the publications based on search terms including genes, mutations and disease. The present disclosure describes a platform for fulfilling advanced research needs, and includes research tools for analyzing various genetic loci. Surprisingly, this sophistication of next generation analysis tools will provide innovative and sophisticated capabilities that will help clinical researchers, and clinicians at the point of care, with deeper insights for improved diagnosis and treatment of diseases.

### SUMMARY OF THE INVENTION

Embodiments describing systems and methods for the analysis of features, mutations, and their effects in the genome that are responsible for wellness and disease are presented. In one embodiment, methodologies for the identification of statistical features of disease-causing mutations that are published in the literature, through novel ‘statistical graphing’ approaches, are provided. These features are employed in building algorithms for determining the deleteriousness and disease causality of a genetic mutation. This system is termed Gene Disease Mutation Analysis Platform™ (GDMAP™).

In some embodiments, a method of analysis of features, mutations, and genomes is presented, the method comprising receiving a plurality of nucleotides comprising a genetic element in a gene, calculating the frequency of mutations of a nucleotide at a position within the genetic element, wherein the nucleotide at the position within the genetic element is replaced by an alternative nucleotide, calculating the total number of mutations for the sequence length of the genetic element, and calculating a deleteriousness score for each position based on the frequency of mutations.

In some embodiments, a method of analysis of features, mutations, and genomes is presented, the method comprising collecting one or more publications, wherein the one or more publications are associated with data comprising genes, and mutations, or diseases, identifying the data comprising genes and retrieving at least one genetic element, wherein the at least one genetic element comprises a 5′ UTR, a promoter, an enhancer, a silencer, an exon, an intron, a coding sequence, a non-protein coding RNA, a splice acceptor, a splice donor, a branch point site, a 3′-UTR, a Kozak sequence, or a poly-A addition site, determining the similarity scores of each of the genetic elements, comparing the similarity scores of genetic elements with a reference sequence for the corresponding element, and assessing the effect of mutation or disease.

In some embodiments, a method to analyze data sets to combine genetic features and compare similarity scores to one or more genetic elements serves to identify real exons in an uncharacterized genomic sequence. The product is a complete gene comprising of consecutive exons that would lead to a complete protein. This system is named SpliceCodeTM

In some embodiments, a computer implemented method for comparing similarity scores is presented, the method comprising receiving a nucleotide sequence from a reference genome comprising at least one genetic element, wherein the at least one genetic element is selected from a list comprising: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof, determining a first exon from the nucleotide sequence, wherein the first exon begins with an initiator codon, wherein the first exon ends with a donor sequence, determining a middle exon from the nucleotide sequence, determining a last exon from the nucleotide sequence, and annotating splicing and regulatory elements based on position weight matrix scores or similarity scores.

In some aspects, the techniques described herein relate to a method of analysis of features, mutations, genes, and genomes, the method including: receiving a plurality of nucleotides including a genetic element in a gene, wherein the plurality of nucleotides are assigned a position, wherein the plurality of nucleotides are arranged in a sequence; calculating a frequency of mutations for each position within the genetic element based on publications, wherein the nucleotide at the position within the genetic element is replaced by an alternative nucleotide; calculating the total number of mutations for the sequence length of the genetic element; and generating a deleteriousness score for each specific position based on the frequency of mutations at that position relative to the total number of mutations.

In some aspects, the techniques described herein relate to a method for identifying a gene in a raw DNA sequence, the method including, receiving a nucleotide sequence from a reference genome, the reference genome including at least one genetic element, wherein the at least one genetic element is selected from a list including: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof; identifying a first exon from the nucleotide sequence, wherein the first exon begins with an initiator codon, wherein the first exon ends with a first donor sequence, and the first exon is bounded by an open reading frame (ORF); identifying one or more middle exons from the nucleotide sequence, wherein the middle exon starts with a first acceptor sequence and ends with a second donor sequence, and the middle exon is bounded by the open reading frame (ORF); identifying a last exon from the nucleotide sequence, wherein the last exon starts with an acceptor sequence and ends with a stop codon, and the last exon is bounded by the open reading frame (ORF); and, annotating the splicing and regulatory elements within the gene based on similarity scores or position weight matrix scores.

In some aspects, the techniques described herein relate to a computer implemented method, including, receiving a nucleotide string including at least one genetic element, the at least one genetic element selected from one of: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof, from a known protein coding gene, or a regulatory, splicing, or functional element of a non-protein coding RNA gene from a reference genome; generating one or more modified nucleotide strings, wherein each base on the one or more modified nucleotide strings is replaced compared to the nucleotide string, wherein replacing each base includes converting each base to a non-identical nucleotide; for the at least one genetic element, calculating the similarity score of the element for every one of the one or more modified nucleotide strings; determining overall deleteriousness by comparing the similarity scores for the at least one genetic element for every one of the one or more modified nucleotide strings and for the nucleotide string; assigning a molecular effect, the molecular effect selected from one or more of: abolition, reduction or enhancement of transcription or translation, exon skipping, intron retention, cryptic exon creation or partial exon deletion due to the deleterious mutation; and storing the information of the molecular effect for every one or more modified nucleotide strings in a memory.

In some aspects, the techniques described herein relate to a computer implemented method for automatically assessing genomic features including: receiving an input dataset including one or more regulatory and/or one or more splicing elements in a gene set; generating one or more similarity scores for the one or more regulatory and/or one or more splicing elements; generating one or more pathogenic or strength altering mutations, wherein generating one or more pathogenic or strength altering mutations involves calculating pathogenicity of known mutations in the one or more regulatory and/or one or more splicing elements, and the difference between the scores before and after mutation; training an artificial intelligence program with the one or more regulatory and/or one or more splicing elements, wherein the one or more similarity scores are within a preset range; training the artificial intelligence program with known pathogenic or strength altering mutations in splicing or regulatory elements in a set of genes with known splicing and regulatory elements, genomic positions, and similarity scores; generating an output dataset of splicing or regulatory elements, wherein the input dataset includes a new set of genes; and generating pathogenic or strength altering mutations for the new set of genes.

In some embodiments, one or more rare variants or mutations in one or more genetic features in one or more genes in a genome that would have an effect on the processing of a gene into a protein, such as transcription, splicing, transport of mRNA from the cell nucleus into the cytoplasm, and translation into the protein are identified, from among one or more variants that occur in the human population. Advantageously, the described method may lead to rapid identification of causative genes and mutations that lead to disease and drug response phenotypes from a patient. This system is named Rapid Whole Genome Interpretation™ (rWGI). In some embodiments, described herein is a computer implemented method for interpreting a genome comprising, receiving a nucleotide string comprising at least one genetic element, wherein the at least one genetic element comprises: a 5′-UTR, a promoter, an enhancer, a silencer, an exon, intron, a coding sequence, a non-protein coding RNA, a splice acceptor, a splice donor. a branch point site, a 3′-UTR, a Kozak sequence, a poly-A addition site or signal, or a cryptic version thereof, for each base in the nucleotide string, generating at least one alternative nucleotide, thereby generating at least one alternative nucleotide string, wherein for each base in the alternative nucleotide string, the nucleotide differs compared to the same position of the nucleotide string, calculating a similarity score for the at least one genetic element for the nucleotide string, and all alternative nucleotide string(s), and calculating downstream molecular effects,

In some embodiments, AI/ML systems are trained to recognize certain sequence and structural features for gene regulation (gene expression), splicing, mRNA transport, and translation. The described AI/ML systems are also trained to recognize alternative genome features due to mutations at one or more positions of genetic elements within one or more genes with or without relevance to disease causation. The trained systems and models are applicable in real patient data to reveal genetic causes of disease and drug responses. Various steps of the AI/ML training, testing and arriving at the implementable models with various objectives are described. This system is named Genome Artificial Intelligence (GenomeAI™). These and other embodiments are described in more detail below.

### BRIEF DESCRIPTION OF THE DRAWINGS

FIG. 1A illustrates the distribution of mutations at different sequence positions of a genetic element, say donor.

FIG. 1B illustrates the distribution of mutations at different sequence positions of a genetic element, say acceptor.

FIG. 2 illustrates the distribution of pathogenic mutations changing from one base into other bases in the donors of genes.

FIG. 3 illustrates the distribution of pathogenic mutations changing from one base into other bases in the acceptors of genes.

FIG. 4 illustrates the position weight matrix of a particular genetic element, donor.

FIG. 5 illustrates the position weight matrix of a particular genetic element, acceptor.

FIG. 6 illustrates the distribution of different types of regulatory and splicing aberrations caused due to mutations in regulatory and splicing elements in various genes that are reported in publications.

FIG. 7 illustrates the distribution of mutations within TP53, a known oncogene, collected from published articles in the literature.

FIG. 8 illustrates the distribution of mutations within a particular genetic element within TP53.

FIG. 9 illustrates the relative frequency distribution of publications regarding differing genetic elements in various diseases.

FIG. 10 illustrates pathogenic mutations occurring in the non-allowable region of the variable amino acid sequence signature.

FIG. 11 is a block diagram depicting an embodiment(s) of a computer hardware system configured to run software for implementing one or more embodiments of systems, devices, and methods for genome analysis.

FIG. 12 is an embodiment of a method to analyze and visualize one or more genetic elements.

FIG. 13 is a block diagram depicting an embodiment(s) of a computer hardware system configured to run software for implementing one or more embodiments of systems, devices, and methods for genome analysis.

FIG. 14 is an embodiment of a method to analyze and visualize one or more genetic elements.

FIG. 15 is an embodiment of a method to analyze and visualize one or more genetic elements.

### DETAILED DESCRIPTION

Searching for the genes and mutations that cause a disease is only the first step in understanding the disease. Enabling the understanding of how a mutation causes a gene to become defective, and how this defective gene leads to disease, can be significantly more beneficial to decipher the causation of a disease. Moreover, differing publications describe different genes and mutations that cause different diseases. Depicting the mutations on the structure of a gene on the sequence of the corresponding elements such as the coding sequence and the different elements representing the regulatory and splicing processes, and computing the frequencies of the molecular changes due to mutations in the various genetic elements and their sequences, may reveal important insights. These insights will be key to understanding the involvement of the defects in different elements in their ability to cause the gene to become defective, resulting in a defective protein that ultimately leads to disease.

Mutations in different elements cause different molecular effects to the primary RNA transcript, spliced mRNA, and the protein, during the different steps of transcription, splicing, polyadenylation, transport of the mRNA from the nucleus to cytoplasm, and the translation of the mRNA into a protein. The tabular and statistical-graphical (graphstats or stat-graph) depiction of the particular mutations on the structures and sequences of the different elements of a gene will enable the deeper understanding of disease causation. The analytical and visualization capabilities of the structural and functional implications of the mutations and their aberrations on the transcripts, mRNA, and protein, will be a boon to the researchers and practicing clinicians in achieving their goals of understanding the disease causation. Such a system will further enable the comprehension of the disease processes in a given patient with a particular disease, and aid in the precise diagnosis and treatment of disease.

In addition to representing the statistics of mutations on the different elements on the gene structure, described herein is a system to represent the statistics of the different mutations in the different sequence positions of every genetic element in described genes, populated from the collected data of disease causing mutations from across a large population of patients exhibiting different diseases from scientific publications. Further, determining the various methodologies in which the different mutations in the different elements of a gene lead to the aberrations in the various steps of gene expression regulation, transcription initiation and termination, splicing, polyadenylation, mRNA transport, translation initiation and termination, and other processing steps of the primary transcript and the mRNA, will be immensely helpful in understanding the causation of every disease, therapeutic effect, and pharmacogenomic indication.

The present system therefore determines the scores and assesses the pathogenicity of each of the mutations from each publication, using several algorithms such as the Shapiro-Senapathy, MaxEntScan, and NNSplice scoring, as well as their modifications and combinations thereof, and the molecular aberrations within the gene, transcript and protein. The system collects data for all of the molecular effects and aberrations for each gene and for each disease, and provide tools to analyze them through statistical and graphical (stat-graph) methods.

Described herein is a system, or platform in which the details of identified mutations will be overlaid on the different genetic elements in a given gene in various graphical and statistical ways. The platform determines if and how a mutation within the sequence of each genetic element causes an aberration in the transcription of a gene, splicing of the primary RNA transcript into the mRNA copy, polyadenylation of the mRNA, or the translation of the mRNA into the protein. Algorithms described herein are used to determine the possible aberration caused by a mutation in each type of the genetic element within a gene at each step of the genetic processes.

For example, a mutation in a donor splicing element in a particular exon of a specific gene such as TP53 can cause an exon skipping or intron inclusion during the splicing process, or premature termination of the mRNA into protein. The platform depicts the mutations on the elements, and illustrates in various ways a graphical animation of the erroneous processes both in the gene structure and in the sequence representation of the whole gene. In one embodiment, the system also shows the defects created in the protein sequence and in a 3D structure of the protein when available. Furthermore, the platform depicts several details of the genetic elements, when a mutation that causes a genetic defect occurs. The platform has the ability to analyze each mutation from a publication based on its several algorithms for different genetic elements, to categorize the pathogenic, strength altering, and non-pathogenic mutations, and to identify the potential molecular aberrations.

Therefore, the system allows for researchers to perform comprehensive meta-analyses by the collective analysis of published mutations of all the genetic elements in the genes causing various genetic and protein aberrations, leading to various diseases in a large number of patients, in a combination of statistical and graphical approaches than the analysis of individual mutations.

Pathology of a gene indicates its biology, depicting where in the genome biology the particular gene participates. The pathology of the mutations in a genetic element through the collective statistical and graphical (stat-graph™) approach, based on a large number of mutations within a genetic element, will indicate the genetic and biological environment that the element is involved in. This approach to connect the biology and pathology of the individual genetic elements through the statistical-graphing analysis will be able to uncover deeper insights into the molecular causation of the disease.

### Predicting the Frequency Pattern of Published Mutations across the Variable Sequences of Genetic Elements

A sequence for a particular type of splicing element such as the donor varies across different exons of a particular gene, and across different genes in a genome. These variations are represented by the frequency of different bases (or nucleotides) at every position of the sequence representing the element throughout the genome, which culminates in the Position Weight Matrix (PWM) of that element. This PWM defines the range of variations that can occur within the sequence of a particular type of element, although there can be a small minority of sequences significantly varying from the PWM across the genes within the human genome.

Many variants that change the sequence of the element in the individuals of an organism, can affect the function of that element, causing an aberration in the transcription or splicing of the transcript, and leading to a disease. However, some of the variants can lead to increased or decreased function of the element, causing a slightly changed binding affinity of the element to their target binding proteins or other molecules such as the small nuclear RNAs (snRNAs), and lead to an increased or decreased transcription, splicing or translation. Such variants may or may not cause a disease state, or at least increase one's predilection towards developing a certain disease state. Herein, the described system may be configured to predict that the frequency of the disease causing pathogenic mutations that occur within the sequence of an element across numerous patients should closely represent the sequence variations associated with the element depicted in its PWM.

Mutations of a type of genetic element (e.g., donor splicing) at different locations within a gene (e.g, in the different exons of a 20-exon containing gene) will affect the function of that element differentially. Mutations in different locations may have different levels of deleteriousness or pathogenicity due to the genetic environment in which they are present. A mutation in one location may be more pathogenic than a similar mutation in another location. This will be revealed by the statistical-graphing method of GDMAP by the frequencies of mutations in a type of genetic element in different locations within a gene across various patients.

Herein, the described algorithms can additionally predict the aberrational effect, such as a splicing effect including exon skipping, intron inclusion, partial exon deletion, or cryptic exon creation, for every possible mutation within every possible genetic element within every gene. The system is then capable of correlating the aberrational consequences of disease causation (or drug response phenotype) of published mutations for every sequence position within every genetic element of every gene with the predicted molecular aberrations. The system will then be capable of revealing the different positions exhibiting various genetic elements in the gene that are differentially capable of causing disease when a mutation occurs in them. This will aid in predicting the aberration and disease-causality of a new mutation that arises in a new patient that was unknown before from the literature.

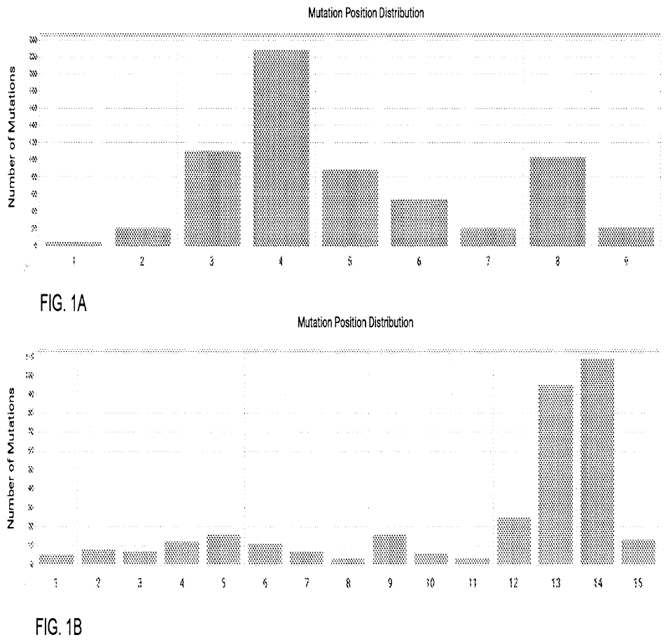

FIG. 1A and FIG. 1B illustrates the distribution of mutations at different sequence positions of a genetic element, for example, in each position within the sequence of acceptor (FIG. 1B) and donor (FIG. 1A).

### The Statistical Distribution of Published Mutations on the Variable Sequence Matrix of Every Genetic Element and Their Disease Implications

In some embodiments, the frequency distribution of the published mutations across the different sequence positions of every type of element is plotted without regard to individually published genes or diseases. Therefore, the frequency distribution of the published mutations across the different sequence positions of every different element within a given gene is plotted, whether the gene is part of a panel of genes implicated in a particular disease or not. It is expected that when an increasing number of mutations are plotted from an increasing number of publications, the frequency of mutations in the different base positions within the sequence of an element increases, and reaches a maximum for one or more sequence positions.

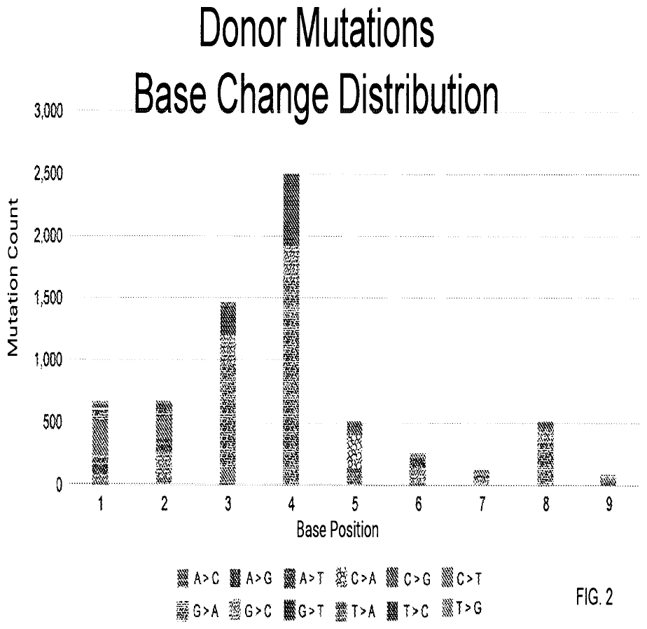

The frequency pattern of a normalized graph for this distribution will be characteristic of the frequency of the bases in the different positions reflecting the disease causality of the various positions, as illustrated in FIGS. 2-5. Although this may closely resemble the PWM for the corresponding element, there are key variations that would help in determining the deleteriousness and disease causality of a type of mutation at a particular position within a genetic element. Using these characteristics of the weights of mutations at the different positions of a genetic element, compared to the PWM of that element, a scoring algorithm for deleteriousness of a mutation from a patient was developed.

It can be expected that the frequency patterns of published mutations in different locations of the same type of splicing element such as a donor within a gene will be different. This is because the importance or weightage of the element in the splicing reaction of a particular exon among the multiple splicing elements for that exon will vary for different splice sites for different exons.

We also expect that when published mutations from various diseases are plotted on all of the 20,000 genes on the different genetic elements including the coding, regulatory and splicing elements, they will show the characteristic frequency patterns as predicted above. This pattern will reveal the genes (and the genetic elements with the genes) that are most disease causing and the genes that are the least disease causing across all diseases covered by the publications. This process will also reveal genes that are not disease causing, to the extent that the publications cover all of the human diseases and all of the genes.

In addition, the same type of frequency distribution from a particular disease will reveal the genes that are causal of that disease. Further, this set of genes might include some novel genes that are not included in the panel of genes that has been thought to be causal of the disease. A score is assigned to the genes based on the frequencies of the mutations in the different genetic elements and in the different genes for the causality of a particular disease, which can be used in predicting a disease in a patient.

### Differential Base Changes at Every Sequence Position of a Genetic Element

The platform shows that when the different bases at the different sequence positions within a genetic element are mutated, they mutate differentially to the other 3 bases. For instance, the G at position +2 of the donor splice site mutates most often to A, than to other bases. Based on these frequencies, described herein is a scoring formula for pathogenic mutation for every base change at each sequence position of the donor, which could reflect a measure of disease causality. Thus, even between the two canonical bases within a splicing element such as the donor, the disease causality may vary for some biological reasons that are not immediately apparent.

Based on PWM, the mutations at the two canonical bases of the donor splice site should lead to the same level of deleteriousness and thus disease causality. As such, the unique variations of specific base changes at the different sequence positions of a splicing element observed in the analysis aids allows for a unique way of determining the deleteriousness of a mutation that could be a measure of disease causality, which should apply for any genetic element collectively in the human genome, as well as individually for any genetic element in each of the genes in the human genome.

We also predict that we can create a PWM of the CDS of a gene, based on mono, di, tri, and oligonucleotides or oligo amino acids that occur in different individuals (with or without a disease). It will form variable amino acids (AA) or nucleotides at many positions. The frequency that we will obtain from the published mutations will follow these patterns. In addition, the frequency of published mutations at the different AA positions will reveal the AAs to which an AA at a given position would mutate most frequently. We have formulated an algorithm to assign scores for the mutations from an AA at every position of the variable AA signature of a domain into the different AAs based on the frequency of the AAs that are mutated into.

We have determined that the frequencies of mutated AA changes will reflect the inherent frequency of different amino acids that could occur within the set of variable (or allowed) amino acids at a given sequence position of a protein domain. However, they may change in unique ways due to so many other parameters and biological or biochemical environments, such as the requirement for co-occurrence or co-dependence of amino acids at different positions of the variable amino acid sequence signature of a domain or protein.

### Differential Frequency of Base Changes at Every Sequence Position of a Genetic Element

The platform shows that when the different bases at different sequence positions within a genetic element are mutated, they mutate at different frequencies to the other 3 bases. The analysis shows that, for instance, the G at position +2 of the donor mutates most often to A, than to other bases. This indicates that the G→A change at this specific sequence position in the donor splice site alters or diminishes the function of the donor splice site in such a manner to cause a severe aberration of the splicing process of the corresponding gene, leading to the disease more often than the change to the other two bases. Based on these variable mutation frequencies at different sequence positions, the scoring formula that we defined would indicate the pathogenicity or deleteriousness of a mutation and its disease causality at each position of the donor.

The variable frequency pattern of mutations will be characteristic of a particular genetic element within a gene, and will be different for different elements within the same gene. Thus, this pattern can be considered to be a Mutational Frequency Position Weight Matrix (MFPWM) for every element within a gene computed or defined based on its consensus sequence. A patient mutation that occurs within a particular element in a particular gene can be scored based on this MFPWM.

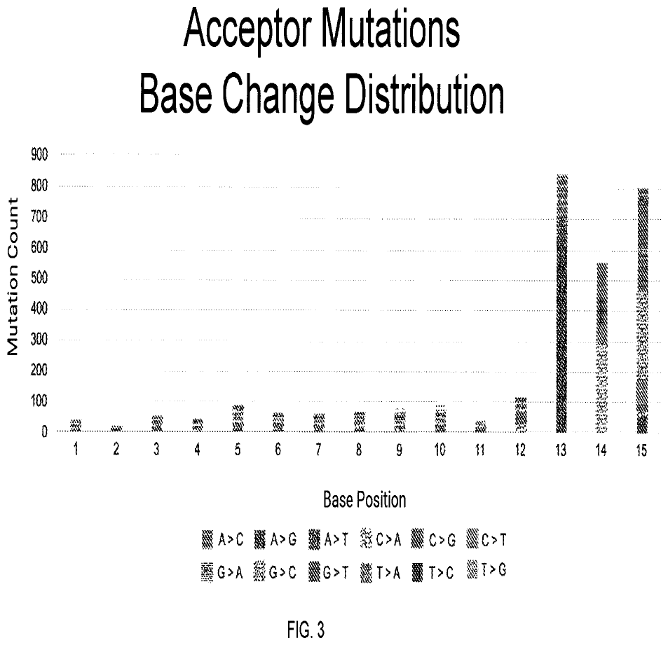

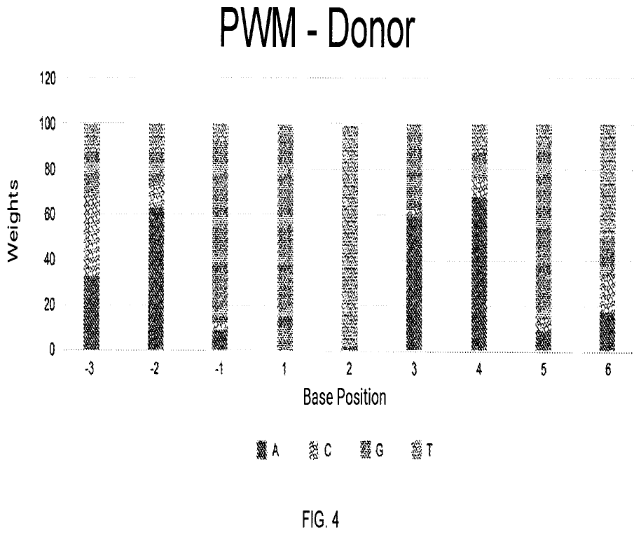

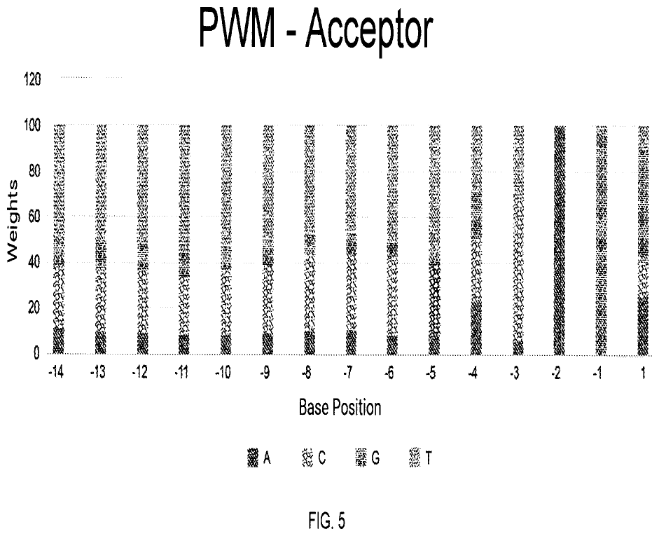

The frequency of base changes at every position of the donor (FIG. 2) and acceptor (FIG. 3), from one base into the other three bases, collected from different publications are plotted. The figures (FIGS. 4 and 5) show the frequencies of bases that occur in the PWM of donor and acceptor, respectively.

### Mutations within the Regulatory Elements and Motifs

It is known that promoter elements occur at variable distances from the transcription start site in different genes, upstream, within or downstream of the genes. For the majority of the genes in a genome including the human, the exact location of all promoter elements is not known. The depiction of the frequency and statistics of the disease causing mutations on the gene structure in tabular and graphical ways in different genes may reveal the important areas of the promoters and the actual base changes into other bases. These important features will not be revealed from the knowledge of mutations at given individual positions or in individual patients. However, when a large number of mutational data from a large number of patients are depicted in various statistical and graphical approaches and various analytical and graphical methods are applied to them, it will reveal the important features of the different promoter elements and motifs in causing a disease. This can be done for every promoter element collectively that occur throughout the genome.

This is true with the mutations that occur within the enhancers and silencers of the gene regulatory elements and the splicing elements. In addition, this type of collective statistical and graphical analysis from published data will reveal the mutational characteristics in the different elements, their sequence features and the sequence positions at which they occur within every gene. Thus, this type of analytical capability will reveal the biological and clinical implications of mutations in various features and elements in every gene, and may reveal novel elements and genes in a genome. The novel algorithms that we described above for the splicing elements also apply to the promoter elements and other gene regulatory and splicing enhancers and silencers.

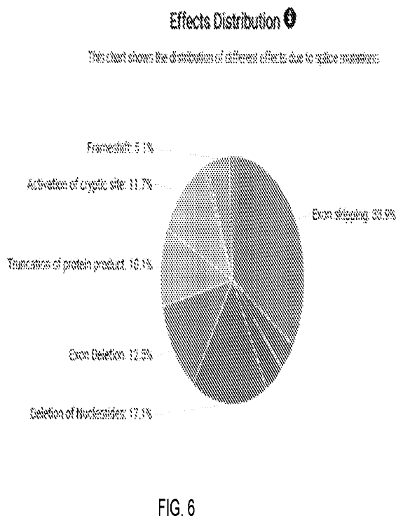

FIG. 6 illustrates a distribution of molecular effects due to mutations in different genetic elements. The distribution of molecular or biological effects such as exon skipping, intron retention, partial exon deletion, or cryptic exon creation, caused due to mutations in genetic elements in different genes from different publications are shown. Likewise, other molecular effects such as abolition, increase or decrease of transcription (gene expression) or translation due to mutations in regulatory elements are also computed. Based on the statistics of the collective effects within a gene from published data, the scoring algorithms will lead to disease causality score for a given mutation from a given patient.

### Mutations in the Coding Sequence (CDS) Regions of Genes

In the current field, mutations that lead to a non-synonymous amino acid (mis-sense), and mutations that lead to a gain of stop codon (non-sense) would be taken as deleterious, and that synonymous mutations are generally non-deleterious. However, there are numerous instances where this is not true, and which needs to be identified to correctly pinpoint the pathogenic mutations. As the amino acid sequence of a protein is not a fixed sequence, the amino acid at a given position can be changed into a set of other amino acids without changing the structure or function of the protein. This set of variable amino acids that do not alter the structure or function of the protein forms the allowable amino acids, and those that alter the structure or function of the protein forms the non-allowable amino acids. It is predicted that the sequence of variable AAs at every position of a domain or protein forms a variable amino acid sequence signature that is characteristic of the domain or protein.

Therefore, pathogenic mutations will occur differently at different positions in the coding sequence of a gene based on the allowed and non-allowed amino acid variability at the different amino acid positions. Mutations that occur among the allowed amino acids at a given amino acid sequence position should be non-deleterious, and mutations that change an allowed amino acid to a non-allowed amino acid will be deleterious. This is different from the difference between a synonymous and non-synonymous amino acid change. Therefore, a system of the present embodiment is able to identify positive and negative amino acid sequence signatures, and the mutations that occur within the positive regions are predicted to be non-deleterious and mutations that occur from a positive region to a negative region will be deleterious.

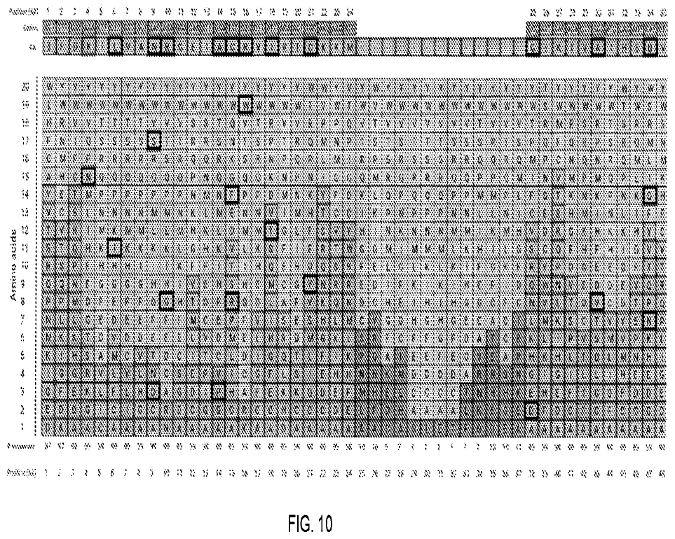

In this scenario, the mutations from patients that are determined to be pathogenic, causal of various diseases should occur in such a manner that the amino acids in the positive signature region should change into the negative signature region. As shown in FIG. 10, pathogenic mutations largely shifted from positive to negative when assessed against published mutations causal of various diseases, and showed a broad correlation as predicted. Any deviation from this prediction could be due to the possibility that the mutation that is reported to be pathogenic in the publication may not be truly pathogenic, or due to an error in the amino acid variability that is reported in the variability data from resources such as the Pfam.

A gene can be involved in the causation of one disease or more than one disease. For instance, the genes that encode DNA binding proteins that regulate the expression of other genes may be involved in multiple diseases. Thus, the GDMAP platform has two ways of depicting the results for a protein, one from each disease separately, and the other from all diseases together. We predict that the variable pattern of mutations at the different amino acid positions within a gene's protein would be the same regardless of the disease, as the gene mutation that causes a disease or any phenotype would lead to a deleterious defect in the protein.

The results can be plotted as a bar chart representing the amino acid positions in the protein (or domain) as the X-axis, and the frequency of mutations that changes one amino acid into various other amino acids from the patients (publications) as the Y-axis. For each amino acid at every position there will be a spectrum of different deleterious amino acids with variable frequencies. The base of the X-axis would represent one of the reference amino acid sequences, and the frequencies of different amino acids that are non-allowed at each of the sequence positions will be plotted on the Y-axis. These non-allowed amino acids and their frequencies will accumulate on the Y-axis as the set of deleterious mutations for every allowed amino acid at a given amino acid sequence position, as the data from different patients accumulate.

The frequency of such mutations for every amino acid at a given sequence position can be plotted separately as a bar chart, where the height of the bar at each position will represent the number of non-allowed mutations at that position. In addition, the frequency of a given amino acid mutating to a specific amino acid also will be plotted, which may indicate the pathogenicity or deleteriousness of the specific type of mutations, for example, a Glu→Asp will be at a higher frequency than a Glu→Tyr. In one embodiment of a scoring algorithm described herein, the differentially mutated amino acids will be given different scores that will be used to calculate and predict the causality of disease or any phenotype.

### Predicting Disease Causality of Mutations in the Coding Regions from Published Mutations

Variable amino acid sequence signatures have peaks and valleys, wherein the peaks are highly variable and valleys are low or invariable. We predict that disease causing mutations majorly occur in the low or invariable regions or valleys. Thus, the distribution of disease causing mutations from a large number of publications on the variable sequence signature of a protein domain will reveal that the high frequency of pathogenic mutations occur in the invariable and low variable positions and a low frequency of pathogenic mutations at positions with increasing amino acid variability. We expect that different amino acids will change at different rates or frequencies to other amino acids at the invariant, low variant and high variant positions.

For instance, a particular amino acid at an invariant or a low variable position (e.g., glutamine) will change to a few other specific amino acids variably by a deleterious mutation, and not to all the other 19 amino acids equally. This differential deleterious or strength-altering change of a given amino acid to other amino acids can be used to predict deleteriousness and disease causality, as we did for the differential base changes that occur in the nucleotide sequences of different genetic elements.

### Effects of Pathogenic Mutations in Transcription, Splicing and Translation

The aim of clinical genomics is to understand the molecular basis of a disease, by identifying the actual defect in the decoding of a gene into a protein or the defect in the protein sequence that leads to a non-functional protein, or an increased or decreased quantity (expression) or the level of function of a protein. These defects can happen in any of the steps of transcription, splicing and translation of the gene into the protein, as described in the following sections.

Genetic Elements of Transcription Regulation

The transcription of a gene is regulated by the promoter that occurs upstream of the gene. Several sequence elements such as the TATA box, CAAT box, and GC box comprise the promoters. In addition, several sequence elements that enhance or silence the transcription of a gene are present upstream of the gene, and also occur in the introns and throughout the gene, and rarely far away on the chromosome or genome. Mutations in any of these elements can increase, decrease or abolish the transcription of a gene, and can lead to disease.

Mutations that occur in the genetic elements participating in gene expression (transcription) from publications are plotted on the promoter elements (TATA box, CAAT box, GC box, initiator box) upstream of the gene and throughout the gene in the normalized gene length pattern, and in actual genes. The pattern showed that the frequency of the mutations are high where these elements occur. In addition, the mutations on the sequences of the different promoter elements occur with differential frequencies, as they occur in the PWMs of each element, indicating the differential importance of the bases within each of these elements, and also the different bases to which these reference bases mutate into.

The enhancers and silencers of many genes are not yet discovered. The distribution of pathogenic mutations from a large number of publications on the gene structure and sequence will reveal these unidentified enhancers and silencers and other novel promoter elements. It is known that multiple transcription factor proteins bind to different sequences such as enhancers and silencers that serve in regulating the expression of the gene. The combination of as many as 20 different proteins or more are known to regulate the expression of particular genes. The approach to determining the frequency patterns of pathogenic mutations from a large number of publications from different diseases will enable the discovery of many of these novel elements.

In the cases where gene expression data (from RNASeq) were available, the gene mutations were correlated with the level of expression of the RNA transcripts. When a mutation indicates that the strength of the promoter would be abolished, the particular transcript of the gene was absent in the RNASeq data. When a mutation in a promoter element indicates that the transcription would increase, the transcript should be present at a higher level, and vice versa. The results indicated this to be true.

### Genetic Elements of Splicing Regulation

In a gene with multiple exons, the potential for a given type of splicing element such as a donor to become defective will vary across the donor element present in different exons. Multiple different elements in combination participate in the splicing together of two consecutive exons, and the elimination of the intron that occurs between the two exons. The strength of each of these elements (specified by, for example, the Shapiro-Senapathy score) vary differently across different exons. Consequently, a mutation in different elements in different exons will have different levels of deleterious effects, and lead to different kinds of aberrations such as exon skipping, intron retention, partial exon deletion, and cryptic or pseudo exon creation. Thus, mutations in a donor in one of the exons may have the highest probability of leading to disease in comparison with the mutations in the donors of other exons in a gene.

In addition, different mutations in different sequence positions within a given element can have entirely different effects. These effects are non-trivial and are produced by a complex process of spliceosomal recognition of the various components of the splicing process, including protein and other factors that enable alternative splicing in different developmental stages or tissue specific gene expression.

Described herein is a method and specific algorithms to identify the different possible splicing aberrations due to mutations in the different positions of the sequence of an element, and in different elements. In addition, the enablement of these defects also depends on the environment of the other elements that participate in the splicing process. The algorithms are designed to understand these environments and correlate them with the mutations and correctly predict the aberrational effect of mutations in the process of splicing.

Using this module, the effect of any given pathogenic mutation in any of the splicing elements in a gene can be predicted, and the complete process can be graphically illustrated. The GDMAP platform enables the identification of the pathogenic process of every mutation leading to a protein defect, or increase or decrease of protein production, from the published data for every disease. It also enables the statistical and graphical elaboration of a variety of details for the aberrations caused by every pathogenic or strength altering mutation.

### EXAMPLE 1

We tested the relationship between PWM and disease state possibility by calculating the frequency of pathogenic mutations that occur within the sequence of a particular type of splicing element (e.g., the donor) causing different diseases that are individually reported in numerous publications in various patients. FIG. 2 illustrates the relative base change distribution in donor mutation sites along a nucleotide string. PWM scores were calculated and weighted in FIG. 4. FIG. 3 illustrates the relative base change distribution in acceptor mutation sites along a nucleotide string. PWM scores were calculated and weighted in FIG. 5. The results from the curated data set indicated that the frequency of published mutations causal of disease across the different bases of a given type of splicing element reflects closely the frequency of bases that occur at the different positions of its PWM.

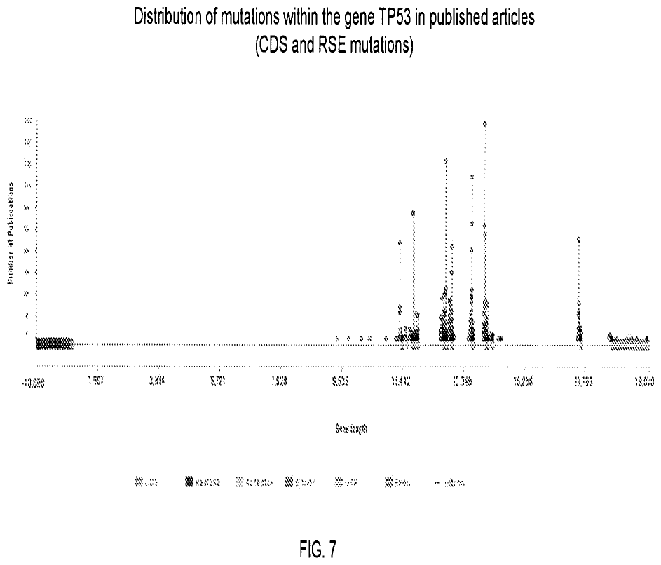

FIG. 7 illustrates the distribution of mutations within TP53, a known oncogene, collected from published articles in the literature. First, a curated data set containing mutations and types of elements from different diseases without regard to the type of disease or the gene was provided.

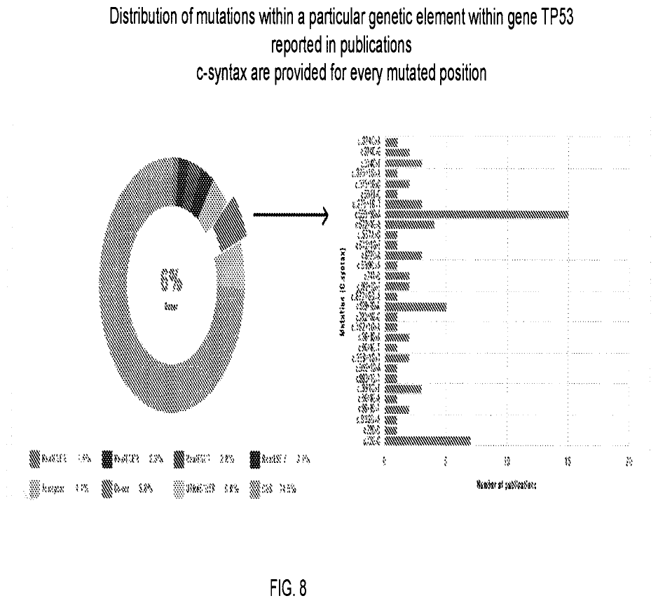

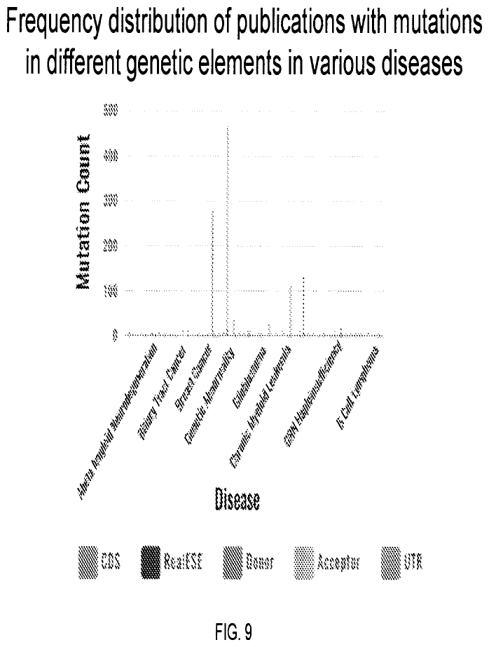

It was predicted that the frequency of pathogenic mutations that occur in a particular genetic element (e.g., a specific promoter or a splicing element) within a particular gene (e.g., the donor splice site in exon 6 of the gene TP53) across numerous individuals exhibiting a particular disease from the published data will represent the inherent sequence variation of that element in that gene across various individuals. FIG. 8 illustrates the distribution of mutations within a particular genetic element within TP53. Moreover, FIG. 9 illustrates the relative frequency distribution of publications regarding differing genetic elements in various diseases. The results produced variable frequencies of mutations at every sequence position of different genetic elements into which a specific nucleotide changed (into the other three nucleotides). This observation is also true when a gene has a deleterious mutation and contributes to the causation of multiple diseases (for example, the gene TP53 that is mutated in many cancers).

It was further predicted that the published pathogenic mutations that occur in the coding sequence in a particular gene across numerous individuals exhibiting a particular disease will represent the inherent amino acid variations in the different amino acid positions across the protein sequence of individuals of that organism, such as that found in the amino acid sequence signature of a protein domain. In this regard, the allowed amino acids at different sequence positions of a protein domain or a protein constitute a positive signature and the dis-allowed amino acids at these positions constitute a negative signature. Thus, when an amino acid is mutated deleteriously causing a pathogenic effect, it will go out of the +ve signature and into the −ve signature, as depicted in the +ve/−ve signature of a protein domain. FIG. 10 illustrates these features by representing the allowable amino acids as the +ve signature (green) and non-allowable amino acids as the −ve signature (red), with mutations overlaid (purple boxes) indicating the pathogenicity of mutations. The reference amino acids are indicated in the blue bar at the top of the grid, and the mutated amino acids are indicated in the red (deleterious) or green (non-deleterious) region within the signature grid.

### Genetic Elements of Translation Regulation

One of the elements that control the translation of a protein from the RNA are the poly-adenylation elements that occur at the tail end of the gene that aids in the accurate addition of poly-A (a stretch of ˜200 As) at the end of the transcript. This set of poly-adenylation elements comprise Poly-A addition signal and site, and the enhancers and silencers. The poly-A sequence at the end of the transcript enables the transport of the mRNA from the nucleus to the cytoplasm where it would be translated. Mutations in these sequences can increase, decrease or abolish this process, which can enhance, reduce or impair the production of the protein.

There are few other elements that enable the translation of the mRNA into protein, such as the Kozak element that occurs around the initiator codon of the mRNA. Mutations in all of these elements from published data indicate that the frequency of the mutations are different at different base positions, and often correspond to the PWMs of the element. In addition, there are upstream and downstream ORFs (uORFs and dORFs) relative to the true start coding of the true ORF within an mRNA, which also are known to be participating in the regulation of translation. Mutations in these elements are also studied by GDMAP in a similar manner.

The enhancers and silencers of poly-adenylation elements of many genes are not yet discovered. The distribution of pathogenic mutations from a large number of publications on the gene structure and sequence will reveal these unidentified enhancers and silencers and other novel translation regulation elements. Determining the frequency patterns of pathogenic mutations from a large number of publications will enable the discovery of many of these novel elements.

Provided herein is a system to identify and score pathogenic mutations sourced from publications as evinced in FIG. 2 and FIG. 3. Broadly, a graph is generated according to the below:
1. Stack the base changes of the mutations (one particular base to any of the other 3 bases) at each sequence position of a genetic element, and each of the different genetic elements occurring within a gene obtained from different publications.
2. When normalized to 100%, the frequency (Y-axis height) of the different sequence positions, and the different elements (e.g., the different donors within the same gene) will raise and keep fluctuating initially.
3. Keep stacking until the frequency of each of the positions gets stabilized.
4. When plots are stabilized, the heights of the different elements of the same type (i.e. donor) at different position within a gene will be different, and the different sequence positions within the same element (donor at a given position, i.e. 7th exon of a 20 exon gene) will differ. The data in this final stabilized pattern will be useful to determine the disease-causality score of each of the base changes at each position of every genetic element, and the different elements within a gene.

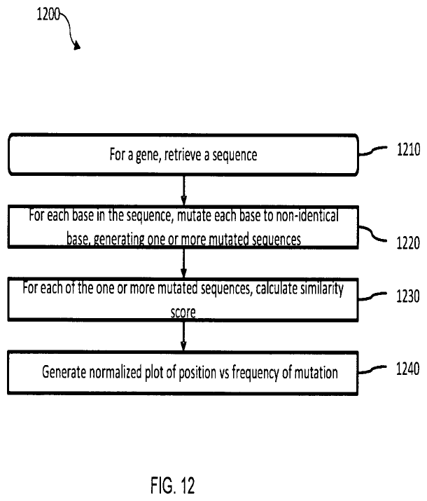

FIG. 12 illustrates an embodiment of the method to identify pathogenic mutations. First, at 1210, the system retrieves a mutation from a selected genetic element for a selected gene from a selected publication. The mutation may be sourced from one or more scientific literature publications, and may include specific annotations. In some embodiments, the annotations indicate known or otherwise tagged genetic elements, such as a promoter, enhancer, or any other known genetic element. At 1220, for each base in the retrieved sequence or mutation, the system proceeds to generate a mutation of one or more bases into a non-identical base, generating one or more mutated sequences. For example, a guanine may be mutated to any nucleotide that is not guanine (i.e. cytosine, adenine, thymine, uracil, depending on whether the selected sequence comprises RNA or DNA). In some embodiments, mutated sequences comprise nucleotides arranged in sequence, wherein the position of one or more nucleotides are mutated into a non-identical base. In one embodiment, an initial nucleotide sequence of 3 bases comprising the sequence AAA, can generate mutated sequences where one, or multiple instances of A are replaced by a U, T, G, C, or any other extant nucleobase besides A. At 1230, for every mutated sequence, the system is configured to use similarity sorting algorithms to sort and categorize pathogenic mutations. In some embodiments, the sorting algorithms are configured to filter out non-pathogenic mutations. At 1240, the system proceeds to generate a plot, with base pair position along the X axis, and “stacked” mutations on the Y axis, as illustrated in FIGS. 2 and 3. The differential base changes (from filtered deleterious mutations) at the different sequence positions of a type of genetic element such as donor generally follow the base frequencies within the PWM in general. However, they vary from the PWM positions in certain ways. For instance, there is a difference between the 2 canonical base positions. The calculation of pathogenicity based on the scores based on PWMs will treat both canonical positions equally. However, if the base-change frequency pattern from the known (published) pathogenic mutations is used in the deleteriousness and disease-causality score determination, these scores will be different and will reflect the biological and pathological reality in the patients. Thus, this type of scoring will better accurately pinpoint the disease causality of a specific mutation than the PWM based scores.

Embodiments of the present disclosure define the genome as the regions within the genome that include the introns in the currently known genes and the intergenic regions between the currently known genes. In some embodiments, a platform as disclosed herein identifies potential genes, protein-coding sequences, and the regulatory regions of these protein-coding genes, as well as the non-coding RNA genes. Accordingly, some embodiments applied the functionalities of multiple modules therein on these newly discovered genes and obtained the various details for CDS and regulatory genetic elements, and their cryptic versions that occur within these genes.

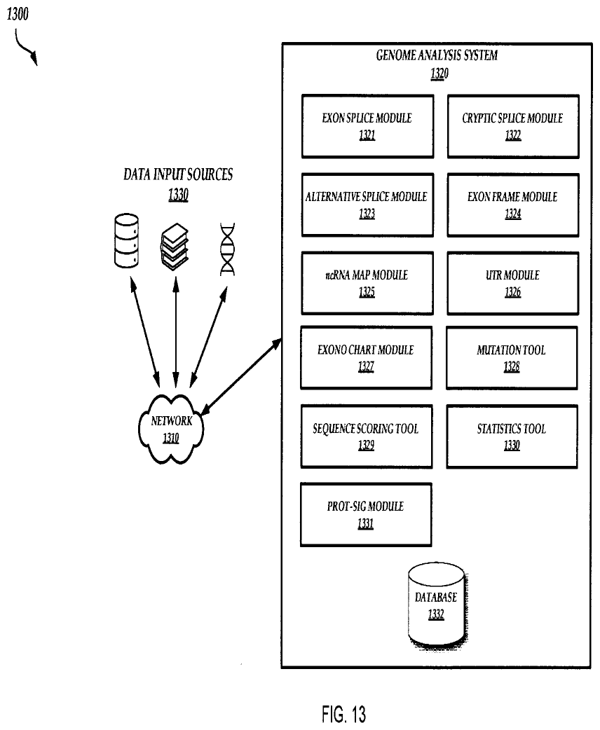

FIG. 13 is a block diagram 1300 illustrating an example genome analysis system 1320, according to certain aspects of the disclosure. Data input sources 1330 are connected via a network 1310 to the genome analysis system 1320. In some embodiments, genome analysis system 1320 may include modules and tools, as well as database 1332, which operatively stores instructions and data received from data input sources 1330. In some embodiments, data input sources 1330 comprise any known scientific journal, repository of scientific literature, primary, secondary, or other sources of genomic and genetic data. In some embodiments, the data collected from the data input sources 1330 comprises gene sequence data, including genetic sequences, specific annotations, and other metadata related to genetic information. The sequence scoring tool 1329 parses at least a portion of a nucleotide string from a genome to identify a splicing site therein. More specifically, sequence scoring tool 1329 identifies, in a nucleotide string, at least one exon, at least one acceptor, at least one donor, and at least one intron between the at least one exon. In some embodiments, sequence scoring tool 1329 may include identifying, in a nucleotide string, at least one exon, and at least one intron between the at least two exons, and a promoter sequence. In some embodiments, sequence scoring tool 1329 may include identifying, in a nucleotide string, a poly-A addition site, wherein the poly-A addition site includes a poly-A site and a signal. In some embodiments, sequence scoring tool 1329 may include identifying a first amino acid string corresponding to a functional protein or protein domain. The Mutation tool 1328 generates mutations in base pairs according to an input sequence, mutating for every base pair at positions upstream and/or downstream from an arbitrary position. In some embodiments, mutation tool 1328 may access a mutation log in database 1332, to identify a recurring mutation over a cohort or a population of individuals. In some embodiments, mutation tool 1328 may identify, in a nucleotide string, a mutation that changes an amino acid to another allowed amino acid (within the positive signature), and a mutation that changes an amino acid to a non-allowed amino acid (within the negative signature) in the functional protein. In some embodiments, mutation tool 1328 determines a deleterious effect of a mutation based on whether the mutation occurs within the positive signature or the negative signature in a protein domain. In some embodiments, mutation tool 1328 identifies, in a nucleotide string coding a protein domain in the functional protein, a mutation leading to a disallowed amino acid. In some embodiments, mutation tool 1328 determines a mutated hydropathy signature of the protein domain based on a hydropathy index of a mutated amino acid. In some embodiments, mutation tool 1328 determines a normal hydropathy signature of the protein domain based on a hydropathy index of an allowed amino acid or a disallowed amino acid, and a deleteriousness score for the mutation based on a difference between the mutated hydropathy signature of the protein domain and the normal hydropathy signature of the protein domain. In some embodiments, mutation tool 1328 also determines a deleteriousness score for the mutation based on whether a mutation occurs within a positive signature indicating no deleteriousness or a negative signature indicating a deleteriousness.

Statistics tool 1330 may perform a frequency analysis over the splice sites and the mutations identified by sequence scoring tool 244 and mutation tool 246. In some embodiments, statistics tool 1330 may use mutation logs and gene sequencing logs in database 252 to evaluate statistical data on a nucleotide string for an individual or a cohort of individuals, for analysis. The algorithm may be a linear or non-linear algorithm, including a neural network, machine learning, or artificial intelligence algorithm used to identify and score splicing sites (e.g., for sequence scoring tool 244). For example, in some embodiments, the algorithm may include the Shapiro & Senapathy algorithm to score a nucleotide string as a splice site (e.g., a ‘donor’ site or an ‘acceptor’ site), a MaxEntScan algorithm, and an NNSplice algorithm, among others. The algorithm may combine various algorithms including the updated version of the Shapiro & Senapathy algorithm to develop biological probability and impact of the various splicing event data throughout the genome.

The Genome Analysis System 1320 may also include different modules which enable the different applications and aspects disclosed herein. For example, some of the modules include an Exon Splice Module 132, Cryptic Splice Module 1322, Alternative Splice Module 1323, Exon Frame Module 1324, ncRNA Map Module 1325, Exon plot module 1327, UTR module 1326, ProtSig module 1331, and Machine Learning Module 1330. Exon splice module 1321 identifies exons in a nucleotide string, and provides data analysis regarding the proteins and protein domains codified by the exons, and the possible protein isoforms or deleterious effects produced by skipping of one or more exons or domains, amino acid rearrangements and other effects or mutations.

A list of genes from an external database, library, or resource (NCBI, ENSEMBL, and the like) may be downloaded and integrated into Database 1332 to provide the list of genes, exons, coding sequence, 5′ and 3′ UTRs, poly-A signal sequences, promoter sequences, and clinical association of genes with diseases (as sourced from dbSNP, COSMIC, and ClinVar). The exons are classified based on their coding features into 5′ and 3′ noncoding sequences, 5′ and 3′ partially coding sequences, fully coding sequences, upstream open reading frames (uORFs), downstream open reading frames (dORFs), poly-adenylated tails, kozak sequence contents, and various promoter boxes (TATA, GC, CAAT, and initiator), each of which are computed, identified, and tagged.

Cryptic splice module 1322 uses algorithms (i.e., the Shapiro & Senapathy algorithm) to identify cryptic splice sites and cryptic exons in human genes. Cryptic splice module 1322 is a beneficial tool that helps investigate splicing mutations in disease, as Cryptic Splice Sites (CSSs) and cryptic exons are known to be involved in numerous diseases. More generally, cryptic versions of every regulatory element occur within a gene sequence. Furthermore, cryptic exons also occur throughout the gene sequence. Cryptic splice module 1322 identifies one or more of these elements throughout the gene sequence, and displays them in graphical, tabular, and sequence views. Cryptic splice module 1322 also determines the mutations that occur within these elements, and displays the details in various forms of illustrations from a subject sequence data and from various public data sources including dbSNP, ClinVar, and COSMIC. Cryptic splice module 1322 also identifies the cryptic versions of other regulatory elements throughout the gene sequence, and the mutations in them, and provides detailed illustrations in various forms.

The exon plot module 1327 enables classification and analysis of exon lengths and their accompanying splicing features, including unusual exon patterns in distinct genes. In some embodiments, exon plot module 1327 applies an algorithm (i.e., the Shapiro & Senapathy algorithm and other relevant algorithms) to determine the scores of real and cryptic splice sites in the outlier exons and other exons in a gene. In some embodiments, exon plot module 1327 enables the analysis of outlying exons that have highly outlying lengths compared to the other exons in the gene, and their real splice sites, cryptic splice sites, real exons, cryptic exons, branch point sites, enhancers and silencers, and their scores. In some embodiments, exon plot module 1327 displays regulatory elements and their cryptic versions within the outlying exon in graphical, tabular, and sequence views. In some embodiments, exon plot module 1327 enables the graphical depiction of exons with repeated lengths and outlying exons in a gene, and their correlations with the splice donor, acceptor and exons scores, and their DNA and protein sequences, using dropdowns for user selection of these features and their involvement in disease. In some embodiments, exon plot module 1327 enables various searching options using nested search boxes for the user to choose the genes with gene length, CDS length, genes having exon length repetition, exons with outlying lengths, disease associated with such genes, and exceptional genes with these features. In some embodiments, exon plot module 1327 enables the search option for genes from various gene panels such as disease panels, drug metabolizing gene (DMG) panels, the American College of Medical Genetics and Genomics (ACMG) gene panels, and other user given gene panels and enabling the visualization and analysis of any gene provided. In some embodiments, exon plot module 1327 provides the capability to analyze different exon classes based on length, length of the preceding and following exons and introns, and the scores of the acceptor and donor splice sites.

In some embodiments, exon plot module 1327 provides the capability to analyze different sets of exons, each set with the same lengths, and their splice scores, exon sequences, amino acid sequences, and the ability to analyze various parameters such as if the sequences of exons of the same length are similar or different, and determining if the splice site sequences and scores are similar or different. In some embodiments, exon plot module 1327 depicts the real and cryptic splice sites by employing Shapiro & Senapathy and other relevant algorithms and comparing the scores for exons with repeat lengths in genes from any given organism, including the human, in an automated manner. In some embodiments, exon plot module 1327 enables the automated analyses of the many features of an exon chart and providing the tabular, graphical, and sequence representation for the analysis of every gene from any organism including animals, plants, and microorganisms. In some embodiments, exon plot module 1327 classifies and analyzes exons based on their coding features into 5′ non-coding sequences, 3′ non-coding sequences, 5′ partially-coding sequences, 3′ partially-coding sequences, and fully coding sequences for the genes with repeated exon lengths and outlying exons. In some embodiments, exon plot module 1327 characterizes the various exons present in a gene into multiple categories based on their length to identify the exon length repetition, highest exon lengths to signify the “outliers” in a gene, and the exception codons which contain no stop codon, in-frame stop codon, or selenocysteine codon sequences. In some embodiments, exon plot module 1327 creates a repository containing information for genes in a genome such as exon details with the exon length, genomic position of the exons, transcript details, real/cryptic splice donors and acceptors, splicing scores, and enabling the display and analysis of any gene by a query. In some embodiments, exon plot module 1327 enables a search for genes that fit various parameters of exon lengths, gene lengths, outlier exon lengths, exons with the same lengths, non-coding, partial coding and fully coding exon lengths, genes from different gene panels, and genes from different diseases, and determines if any disease correlates with such genes or vice versa, and the ability to analyze these genes in graphical, tabular, and sequence illustrations. In some embodiments, exon plot module 1327 overlays the subject(s)' mutations on the gene with depictions in an exon chart, in graphical gene structure and sequence illustrations in color codes for depicting the features of exons, promoter boxes, 5′ and 3′ UTRs, real/cryptic splice sequences, poly-A site and region, branch point regions, and the ability to analyze them for different parameters of exons provided by an exon chart including the correlation of the subject mutations with gene features. In some embodiments, exon plot module 1327 enables analysis of enhancers and silencers in the outlying exons, especially the first and last exons, to determine if the long lengths are required in order to accommodate these regulatory sequences or signals. In some embodiments, exon plot module 1327 indicates the consequences of a mutation in graphical and sequence illustrations, and plotting subject mutations in a real or cryptic splice and exonic regions, and the known mutations from the different databases such as dbSNP, ClinVar, and COSMIC, and categorized into clinical significance, molecular consequence, variation type and pathogenicity based on the SIFT and/or PolyPhen scores on any gene chosen by the user. In some embodiments, exon plot module 1327 enables the query and analysis of different parameters of genes in an exon plot for the detection and analyses of unusual length repetition patterns and splicing patterns in distinct genes, and possible disease connections.

UTR Module 1326 identifies the various promoter elements, 5′ and 3′ UTRs, poly-A sites, and various possible ORFs such as u-ORFs and d-ORFs, their sub classifications within these based on the specific start and stop codons, and their disease connections. In some embodiments, UTR Module 1326 identifies genetic elements in various tabs for analyzing the properties of promoters and UTRs in transcripts and mRNAs such as: mRNA sequence, splice score and promoter, displays the structure of mRNA transcript of a gene and illustrating and enabling the analysis of the properties of un-translated regions (UTRs) in human mRNA sequences, and enables the classification of exons in the transcript into coding, partially-coding, or non-coding exons, providing splice site sequences, and scores for each of them. In some embodiments, UTR Module 1326 locates any upstream and downstream open reading frames (u-ORFs and d-ORFs) that surround the real ORF (CDS), enables the determination of the Kozak consensus sequences surrounding the start codon, and providing Kozak scores for the identified ORFs in upstream and downstream regions, indicating which ORFs may be turned on in different biological contexts, and depicts the structure and sequence of mRNAs and locates the sequence components such as coding sequence, 573′ UTRs, Poly-A signals, initiator ATG codons, stop codons that are in-frame with one or more ATGs, upstream ORFs (u-ORFs) and downstream ORFs (d-ORFs), and displays four different classes of ORFs in upstream and downstream regions of every mRNA transcript of genes, in tabular, graphical, and sequence views. In some embodiments, UTR Module 1326 illustrates different ORF classes such as u-ORF, r-ORF (real open reading frame), and d-ORF between 5′ and 3′ region of coding exons and depicts the occurrences of start and stop codons on the gene's mRNA and for every ORF classes in a graphical, and sequence view, determines the ORF classes and tabulating the features of them such as ORF type, ORF position, Kozak sequence, Kozak score, stop codon sequence, real stop codon score, and 4-base stop codon score, and illustrating them in graphical and sequence view, displays the splice sites for all the exons in a transcript and computing scores using the Shapiro & Senapathy algorithm and other relevant algorithms, and calculating and displaying the exon scores by taking the average of the acceptor and donor scores, and defines different UTR and exon classes in a transcript, and categorizing them as fully coding exon (FCE), 5′ partially-coding exon (PCE5), 3′ partially-coding exon (PCE3), 5′ and 3′ partially-coding exon (PCE53), 5′ non coding exon (NCE5), and 3′ non-coding exon (NCE3). In some embodiments, UTR Module 1326 identifies real and cryptic promoters and poly-A motifs and elements by adapting and modifying other relevant algorithms such as MaxEntScan, NNSplice, and Human splicing Finder throughout the gene sequence and genes in the genome. In some embodiments, UTR Module 1326 identifies real and cryptic splice sites using promoter and poly-A motifs and elements by adapting and modifying other relevant algorithms such as MaxEntScan, NNSplice, and Human splicing Finder throughout the gene sequences and genes in the genome and identifying the known mutations from databases such as ClinVar, dbSNP, and COSMIC. In some embodiments, UTR Module 1326 identifies real and cryptic promoter and poly-A motifs and elements by adapting and modifying other relevant algorithms such as MaxEntScan, NNSplice, and Human splicing Finder throughout the gene sequences and genes in the genome and identifying the mutations from subjects' genome. In some embodiments, UTR Module 1326 enables various search options using nested search boxes for the user to choose the genes based on number of ORFs, number of promoter boxes, promoter box score, poly-A boxes, poly-A box score, exon classes, disease associated genes, exceptional genes, and other parameters.

Alternative splice module 1323 uses the algorithm (e.g., the Shapiro & Senapathy algorithm and other relevant algorithms) to identify alternative splicing events such as exon skipping, intron retention, and alternative splice site usage in each of the predicted isoforms of the given gene. In some embodiments, alternative splice module 1323 provides a catalog of predicted alternative transcripts in human genes, including those that may or may not genuinely encode distinct proteins. Alternative splice module 1323 identifies unique splicing events in the alternative transcripts when compared with a canonical transcript, such as exon skipping, exon inclusion, intron retention, and alternative splice site usage.

Alternative splice module 1323 maps alternative splice events and their molecular effects in different transcripts of a gene compared with the canonical transcript, which is defined by various methods. In addition, it also maps these details based on constitutive exons defined by various methods. In alternative splice module 1323, differences among transcripts are also correlated with changes in the encoded structural domains, thereby capturing the functional regions of proteins that alternative splicing may normally or deleteriously affect. Alternative splice module 1323 thus simplifies the prediction of the particular transcripts resulting in distinct proteins and distinguishes them with the artifacts of mistaken sequence annotation, which is key to the advancement of the field of clinical genomics and Precision Medicine. In some embodiments, alternative splice module 1323 enables the visualization of known mutations, mutations from individual subjects and cohorts of subjects. In addition to the mutational analysis, alternative splice module 1323 also provides analysis of the domains encoded by different isoforms of a gene in a single view. Thus, alternative splice module 1323 provides insight into aspects of alternative splicing in genes, their impacts on functional domains, and mutational analysis.

Alternative splice module 1323 provides multiple ways to view and analyze alternative splicing events, such as based on gene: The alternative splicing events can be visualized for individual transcripts for the selected gene; and based on clinical association: the alternative splicing events can be visualized for individual transcripts for the genes implicated in the panels for all major cancers and inherited disorders. In some embodiments, alternative splice module 1323 provides alternative splicing events, wherein the user can select a particular transcript of a given gene and explore different alternative splicing events including skipped exons, cryptic exons, exons with alternative acceptor splice sites, exons with alternative donor splice sites, exons with alternative acceptor and donor splice sites, and retained introns together. In some embodiments, alternative splice module 1323 identifies genes based on a number of transcripts (and selects the highest, or one of the highest): Genes having a high number of transcripts can be searched (e.g., ranging from 1-28). The alternative splicing events can be visualized for individual transcripts for these selected genes.

Exon frame module 1324 determines the possible distribution of stop codons and coding exons in a reading frame before and after splicing events. A reading frame is a way of dividing the sequence of nucleotides into a set of consecutive, non-overlapping triplets, where these triplets equate to amino acids or stop signals during translation, which are called codons. In some embodiments, exon frame module 1324 analyzes and verifies that a distance in the nucleotide string between two stop codons while mapping different stop codons should not fall inside an exon region. To verify this, the length of each of the exons and the open reading frame are plotted separately. The exon with maximum length in any transcript should be lesser than the maximum distance between two stop codons in all the reading frames. After splicing, the CDS length should be shorter than the maximum distance between two stop codons. In some embodiments, exon frame module 1324 allows the determination, analysis, and illustration of the exon-intron structures across ORF patterns of a gene and determines the structure of a gene with respective reading frames that contain exons of a gene and the patterns of before and after splicing by constructing an image of the entire split gene, including the exons, introns, splice junction signals, and stop codons that occur within each frame. In some embodiments, exon frame module 1324 streamlines the detection of atypical gene patterns, such as long exons, long open reading frames without annotated exons, or short introns, and illustrates exons and ORFs in a single reading frame of the gene along with their splice sites and scores calculated using algorithm (e.g., the Shapiro & Senapathy algorithm and other relevant algorithms). In some embodiments, exon frame module 1324 represents three reading frames of a transcript, along with all possible stop codons in each reading frame and plotting the coding exons in appropriate reading frames by using the reading frame algorithm.

In some embodiments, ncRNA map module 1325 identifies and illustrates ncRNA genes from the human genome, and their splicing and processing into the mature functional RNA molecules in tabular, graphical, and sequence illustrations, and creates a repository for the non coding RNA genes platform containing all possible information for ncRNA genes in a genome such as exon details with the genomic position of the exons, transcript details, exon length, splicing and maturation processes, and consequences of the mutations. In some embodiments, ncRNA map module 1325 identifies mutations in the non-coding RNA genes by modifying and applying the Shapiro & Senapathy algorithm and other relevant algorithms across the gene and genomic scale from individual subjects and in a cohort of subjects, and enabling the clinicians to correlate the mutations in non-coding RNA genes that drive disease pathogenesis, and identifies mutations in the regulatory elements of the non-coding RNA genes responsible for disease-causing, adverse drug reactions and affecting the efficacy of various drugs in a subject. In some embodiments, ncRNA map module 1325 identifies known disease-causing mutations in different ncRNA genes, and using them to predict or diagnose mutations and diseases from the subject genome, parses the identified mutations in non-coding RNA genes against the curated Genome Explorer proprietary mutation database, enabling to distinguish and categorize the known and novel mutations of non-coding RNA genes reported in the individual and cohort subjects, and identifies structural and functional motifs and elements in the non-coding (nc) RNA genes (rRNA, tRNA, miRNA, snRNA, snoRNA, siRNA, IncRNA).

In some embodiments, ncRNA map module 1325 identifies disease-causing mutations in different ncRNA genes, predicting or diagnosing, mutations and diseases from the subject genome, and known disease-causing mutations in different ncRNA genes, using them to predict or diagnose mutations and diseases from the subject genome. In some embodiments, ncRNA map module 1325 identifies sequence signals for processing different ncRNA genes to their mature forms using the modified Shapiro & Senapathy and other algorithms based on consensus, PWMs, and other relevant parameters for all ncRNA genes, and compares subject ncRNA gene sequences with reference sequences to identify mutations using modified Shapiro & Senapathy and other relevant algorithms based on the score difference between the normal and the mutated signals.

Prot-Sig Module 1331 enables the analysis of selected protein features in a genome, and their aberrations due to mutations that lead to diseases and other afflictions such as adverse drug reactions. It further enables the visualization and analysis of various details including the exon-domain signatures, cryptic splice sites, and the protein signature showing variable amino acids at each position of the domains that provides a deeper understanding of the allowed and non-allowed amino acids of the domains. When a gene is chosen in Prot-Sig Module 1331, coding exons of the selected gene and transcript are displayed with their corresponding domains overlaid as colored lines. Mutations on these coding exons can be visualized by selecting the mutation toggle option. On clicking the domains above their coding exons, domain details, and various types of signatures such as 20 colors, Positive-Negative, Hydro, Cryptic splice, Alternative splicing and Whole protein signature, are displayed for further analysis. In some embodiments, Prot-Sig Module 1331 performs or collects alignment results from a third party database (e.g. , database 252, including the Pfam database), including a seed alignment and a full alignment. In some embodiments, a seed alignment includes a set of manually curated amino acids from the domain sequences from several genomes and thus tends to have a smaller number of amino acids than the full alignment. In some embodiments, a full alignment includes a set of amino acids produced from several genomes that are aligned using Hidden Markov models, and the like.

In some embodiments, Prot-Sig Module 1331 determines, analyzes, and illustrates the protein sequence signatures of a protein and its domains, their associated features such as the hydropathy and splicing, and the clinical and biological impacts of genetic mutations. In some embodiments, Prot-Sig Module 1331 provides a protein chart to determine and illustrate the analysis of variable amino acids in protein-coding sequences under three different tabs: Protein Overview, Cryptic Splice Sites, and Variant density. In some embodiments, Prot-Sig Module 1331 converts the amino acid alignments from Pfam database into amino acid signatures of proteins and their domains, by identifying the variable amino acids and avoiding the redundant amino acids at each position, and by determining if an amino acid occurs at greater than a specific fraction (e.g., 50%) of the aligned positions, thus incorporating a unique algorithm. In some embodiments, Prot-Sig Module 1331 defines an algorithm that identifies the different non-redundant amino acids at each position and includes them as the variable or allowed amino acids at that position, taking into account any position with or in the alignment indicating a gap, whereby a position with a particular frequency (e.g., >50%) of dots is defined as grey regions in the signature.

In some embodiments, Prot-Sig Module 1331 determines and displays the set of non-redundant AAs produced from the multiple sequence alignment (MSA), generating a unique signature of allowed AAs for every sequence position, showing each of the 20 AAs in a distinct color, and defines that the allowed and non-allowed regions of the positive-negative signature of a domain or protein determines the pathogenicity or deleteriousness of a variant by its occurrence in the positive (green) or negative (red) region. In some embodiments, Prot-Sig Module 1331 displays the non-redundant AAs from the multiple sequence alignment (for e.g., Pfam database) in one color (e.g., green), and all other AAs in another color (e.g., red), showing a map of allowed (positive) and non-allowed (negative) AA substitution space across the sequence, indicating variants that may result in a viable or defective protein. In some embodiments, Prot-Sig Module 1331 finds that the deleterious (pathogenic) mutations would fall within the negative region (red) and that the benign or likely pathogenic mutations would fall within the positive region (green), and applying this finding in testing and determining if a given variant is deleterious or not, determines the impact and clinical significance of the mutations based on the occurrence of the altered amino acids within the negative amino acid space or the positive amino acid space, thereby showing the amino acids where the actual mutations occur by color codes, and depicts the signature for the exon encoded domains in color codes based on a hydropathy scale. Prot-Sig Module 1331 displays the hydrophobic AAs in shades of a particular color (e.g., red), and hydrophilic AAs shown in shades of another color (e.g., blue) to create a heat-map of hydropathy. In some embodiments, Prot-Sig Module 1331 determines the secondary structure map of the amino acid signature using standard values of secondary structure, and depicting them in different color codes thus creating a color-coded secondary structure signature, which will change due to genetic mutations from a subject or from gene-mutation databases such as ClinVar, dbSNP, and COSMIC. In some embodiments, Prot-Sig Module 1331 defines the secondary structure map of the amino acid signature using standard values of secondary structure, depicting them in different color codes, thus creating a color-coded secondary structure signature and enabling its illustration against the domain signature for the analysis of secondary structures correlating with signatures and mutations in various amino acids. In some embodiments, Prot-Sig Module 1331 enables the illustration, visualization, and analysis of mutations in the 3D structure of the domain along with the amino acid variability in the allowed or non-allowed set of amino acids and correlating and determining the effects of the mutations in the domain. In some embodiments, Prot-Sig Module 1331 represents the structure of coding exons in a gene by a shape such as an oval or rectangle, and overlaying the protein domains encoded by the exons, as available in Pfam database or predicted by PfamScan, or any other amino acid alignment databases, correlating the clinical association of mutations in the CDS with cancers and non-cancer disorders in a user-driven approach, displaying various details of domains encoded by the exons such as domain identifier (Pfamld), class, start and end position of the transcript encoding the domain, and coding exons using i-icons, mouse hovers, and context-sensitive popups, depicts the variable amino acids in the key regions of human proteins, such as domains and deriving the set of “allowed” amino acids by generating the multiple sequence alignments of diverse genomes, creating a signature of potential amino acid substitutions across the domain, and classifying the signature under different tabs including: 20 colors, Positive Negative, Hydro, Cryptic Splice, Alternative Splicing, and whole protein signature, and illustrates the alignment of amino acids under two different tabs: Seed and Full, and depicting the alignment which contains a set of allowed/curated amino acids in the Seed tab and the alignment which contains the set of amino acids produced by Pfam using Hidden-Markov models in the Full tab. In some embodiments, Prot-Sig Module 1331 computes and depicts the signature of potential amino acid substitutions across the domain in color codes based on the hydropathy (hydrophobic and hydrophilic) index, charge of amino acids, and determining its region and impact on the cryptic and alternative splicing sites, creates and depicts the impression of the known amino acid substitutions or subject(s) mutations that are likely to maintain the structure and function of a given protein region, and the mutations that are likely to destroy the structure and function of the protein thus leading to disease, depicts the exons that encode a domain by overlaying the domains on the corresponding positions of the codon and AA sequences, and various features of domains and proteins against the gene sequence, and enables the selection of different score thresholds to view any cryptic splice sites or cryptic exons that occur within the CDS of different exons in different color codes, thereby identifying the cryptic splice sites and cryptic exons within real exons, whose mutations can disrupt normal splicing leading to defective protein and disease.

In some embodiments, Prot-Sig Module 1331 depicts the positions on the signature in which the human amino acid sequence has a gap, but other genomes have amino acids, shown as a dash in the human sequence, in different color codes, and indicating the positions at which lesser or higher than a specific fraction of amino acids occur with or without a gap (e.g., 50%) in the sequence signature. In some embodiments, Prot-Sig Module 1331 provides toggle options to turn on the mutations to overlay known mutations on the signatures from different databases such as dbSNP, ClinVar, and COSMIC, categorized into clinical significance, molecular consequence, variation type, and pathogenicity based on the SIFT and/or PolyPhen scores, and enabling the illustration of the amino acids, cryptic sites, and its scores in graphical, tabular, and sequence with pop-up boxes, mouse hovers, and context sensitive explanations. In some embodiments, Prot-Sig Module 1331 analyzes cryptic sites and exons within the coding sequence of a protein by determining and depicting the cryptic splice sites and cryptic exons, real splice sites and exon positions and their scores in various color codes and shapes, based on different score thresholds within the coding exon sequences in tabular, graphical, and sequence illustrations, analyzes the alternative splicing of the exons coding for the domains and providing the signatures for the added or skipped region of the exons coding for the domain, and enables the pattern analysis of variations in protein and domain sequence signatures for different transcripts of a given gene. In some embodiments, Prot-Sig Module 1331 displays the number of samples for each variant from the COSMIC database for each domain position, and depicts the positions of a specific variant in a color (e.g., red), and positions with more than one variant are depicted in different colors, for example, as follows: two variants->blue, three variants->green, four variants->yellow (named as variant density plot), predicts the splice sites in the genes of any organism using Shapiro & Senapathy and relevant algorithms in an automated manner. Predicting and assigning the score for cryptic exons based on the cryptic donor and acceptor splice site scores, and detecting which amino acid mutation would make the protein defective based on the mutations from one or more subjects within the protein signature, based on where the mutation falls within the positive or negative amino acid space, and determining which mutations are correctly identified and which are incorrectly identified. In some embodiments, Prot-Sig Module 1331 overlays the subject(s) mutations on the gene, and provides visual and analytical illustrations of the mutations from the subject(s) and known mutations from various gene-mutation databases in graphical, tabular, and sequence views with pop-up boxes, mouse hovers, and context sensitive explanations, enables various search options using nested search boxes for the user to choose the genes based on the domain, number of domains in a gene, families, average AA substitutions, alignment type, disease associated genes, domains using Pfam Identifier, and exceptional genes, and provides various information about the gene and its associated elements such as protein family and domains, ontology information, disease phenotypes using i-icons, mouse hovers, and context-sensitive popups.

In some embodiments, machine learning module 1330 is configured to accept one or more types of input, to generate a suitable output to provide analysis of certain genomic features. According to embodiments described herein, training data sets comprising pre-compiled or pre-annotated genetic elements, mutations, or other meta-data for genomic data, may be processed in order to generate novel identification of under characterized or unknown features.

The Genome Analysis System 1320 may be installed onto a platform, including a server, and perform scripts and other routines provided by Genome Analysis System 1320 to display graphics and generate analysis of genomic features.

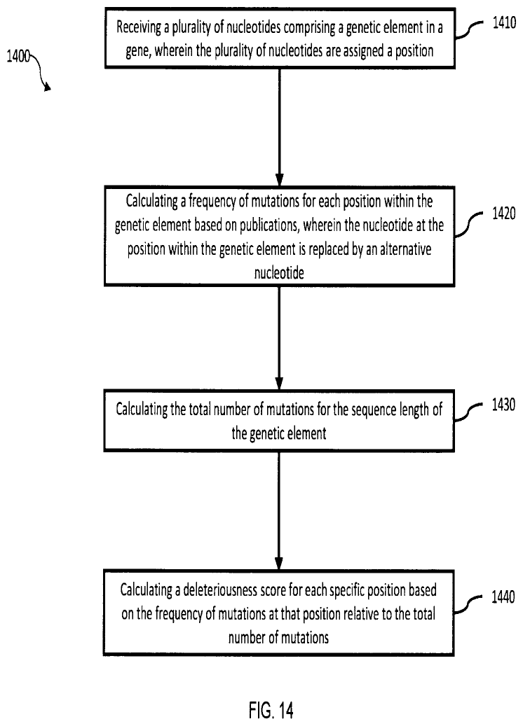

FIG. 14 is a flowchart of an example method for the analysis of features, mutations, genes, and genomes, the method comprising the following steps. At step 1410, the method recites receiving a plurality of nucleotides comprising a genetic element in a gene, wherein the plurality of nucleotides are assigned a position. In some embodiments, the plurality of nucleotides are arranged in a specific sequence. In some embodiments, the plurality of nucleotides are collected from a gene repository. In some embodiments, the plurality of nucleotides are assigned or annotated with genetic elements. In some embodiments, the plurality of nucleotides are collected from one or more scientific publications. At step 1420, the method further comprises calculating a frequency of mutation for each position within the genetic element, wherein the nucleotide at the position within the genetic element is replaced by an alternative nucleotide. For example, a nucleotide sequence of 3 bases comprising the sequence AAA, can generate mutated sequences where one, or multiple instances of A are replaced by a U, T, G, C, or any other extant nucleobase besides A. At step 1430, the method then proceeds to calculate the total number of mutations for the sequence length of the genetic element. At step 1440, the system calculates a deleteriousness score for each specific position based on the frequency of mutations at that position relative to the total number of mutations.

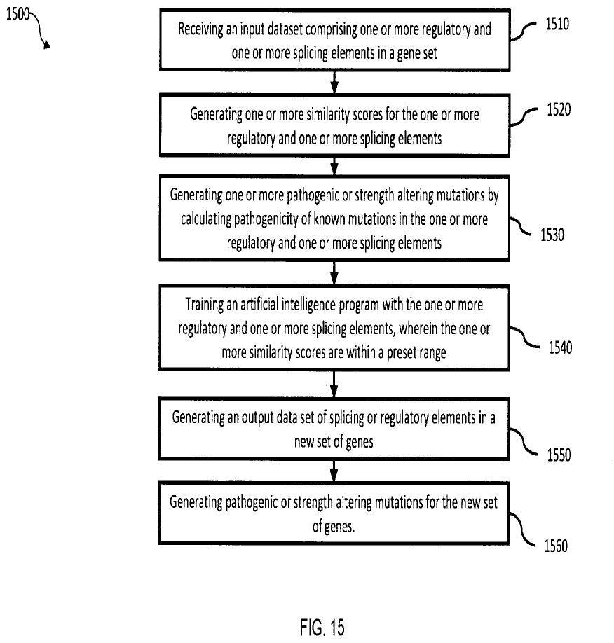

FIG. 15 is a flowchart of an example computer implemented method for automatically assessing genomic features, the method comprising step 1510, receiving an input dataset comprising one or more regulatory and/or one or more splicing elements in a gene set. In some embodiments, the gene set comprises one or more gene sequences, each gene sequence annotated with existing genomic features. Then at step 1520, the method proceeds to generate one or more similarity scores for the one or more regulatory and/or one or more splicing elements. In some embodiments, the similarity scores are calculated using an algorithm as described herein. (i.e. Shapiro-Senapathy, MaxEntScan, or NNSplice scoring). At step 1530, the method then generates one or more pathogenic or strength altering mutations in the gene set by calculating pathogenicity of known mutations in the one or more regulatory and one or more splicing elements. At step 1540, an AI model is trained with a subset of the one or more regulatory and one or more splicing elements of the gene set, wherein the subset of the one or more regulatory and one or more splicing elements are chosen by prossessing one or more similarity scores within a preset range. In some embodiments, the preset range is within 30. In some embodiments, the preset range is within 10, 20, 40, 50, 60, or 100, or 60-70, 70-80, 80-90, 90-100 or below 50, or any value in between. At step 1550, the process then generates an output data set of splicing or regulatory elements based on a new set of genes. In some embodiments, the new set of genes are different than the gene set from the input dataset. At step 1560, the method then generates pathogenic or strength altering mutations for the new set of genes.

### Identifying Pathogenic Mutations in Cryptic Genetic Elements in Genes with Disease Causality Using Statistical Graphing Method

1. Obtain all possible n-mers from the whole genome sequence based on the length of a genetic element using the sliding window method or other methods. For each of the n-mers:
   a. Calculate the similarity scores based on algorithms such as Shapiro & Senapathy algorithm, MaxEntScan algorithm and NNSplice algorithm or their modified versions based on the length and PWMs of the genetic elements, or their average or weighted scores.
   b. Categorize the n-mers based on the similarity score ranges such as 90-100, 80-90, 70-80 and so on, and,
   c. Plot them on a gene structure and sequence graph with frequency of n-mers in each score range.
   d. These n-mers form the pseudo or cryptic elements.

Further, find the similarity scores for a particular element from all archived genes. Next, categorize each element based on the percentage similarity score, including ranges such as 90-100, 80-90, 70-80, 0-100, or any value in between, and plot the similarity scores on a gene structure and sequence graph with the frequency of n-mers in each score range. Frequency distribution of similarity score ranges in each type of the genetic element, such as acceptor, donor, branch point, enhancer, silencer, promoter and polyA in all of the 20,000 genes are thereby determined.

We observed that the majority of the real elements occurring in the 20,000 genes have similarity scores above 70. The frequency distribution of the cryptic elements in every type of genetic element revealed that the majority of cryptic elements have very low similarity scores compared to the real elements. It also showed that increasing the scores actually decreased the frequency of the occurrence of cryptic elements.

Display the cryptic elements of a particular gene on its structure and sequence, based on the score range selected from a dropdown. Cryptic elements with similarity scores within the selected range, in some embodiments, 80-90 from a dropdown, 70-80, 60-70, 50-60, 40-50, or any value in between the preceding values, are displayed on the gene structure and sequence view.

Cryptic exons or pseudo exons are defined by cryptic genetic elements such as cryptic acceptor, donor, branch point, enhancer, and silencer in the gene sequence, where each of the elements is positioned appropriately resembling a genuine true exon. Plot the cryptic exons on the gene structure and sequence view based on the genetic elements with similarity scores within the selected score ranges, and the selected exon length range such as 50-300 bases, and 50-500 bases. These cryptic exons, when activated due to a mutation in any one of the cryptic elements, can lead to deleterious aberrations causing diseases. All the possible cryptic exons within a gene are determined and the details such as exon length, scores, and their ranks are tabulated.

Mutations from publications that falls on the cryptic elements are similarly plotted on the different elements. Their statistics are determined by various methods as described above, also plotting on the different cryptic genetic elements in each gene. The frequency of mutations in certain cryptic elements within a gene could be much higher than others, indicating that they are present in a genetic environment such that these mutations cause aberrations in gene regulation or splicing. Thus, this is part of statistical graphing method to identify specific mutations in particular cryptic genetic elements within each gene, when a large number of pathogenic mutations can be analyzed in this manner.

### Non-Coding RNA Genes and Their Genetic Elements

Although the number of non-coding RNA genes in the human genome is not yet clearly determined, it is estimated that there may be thousands of ncRNA genes. The different types of ncRNAs, such as micro-RNA (miRNA), transfer RNA (tRNA), ribosomal RNA (rRNA), small nuclear RNA (snRNA), small nucleolar RNA (snoRNA), and long non-coding RNA (lncRNA) have different functions. The ncRNA genes are transcribed, and the primary RNA transcripts are processed to remove parts of the RNA sequences, resulting in the processed functional RNA molecules, which then perform their function. ncRNA genes are associated with important biological processes.

Each type of ncRNA includes promoters, exons, introns and their associated enhancer/silencer elements. As with the protein coding genes, the processing of the primary RNA transcript of an ncRNA is aided by recognition sequences that signal the presence of the exons and the introns, such as the Drosha and Dicer for miRNA genes. Mutations in these regulatory and splicing elements of ncRNA genes, as well as the processed functional RNA, can cause many diseases.

The ncRNAs have now emerged as important players in the diagnosis and therapeutics of many diseases such as cardiovascular diseases (atherosclerosis, cardiac fibrosis, hypertension), neurodegenerative diseases (Spino-cerebral ataxia type 7, Spino-cerebral ataxia type 8, Spinal muscular atrophy, Opitz-Kaveggia syndrome, etc.) cancers (cervix, breast, lung cancer), immune-mediated diseases, and developmental diseases.

The publications concerning diseases caused by ncRNA genes and the causal mutations are emerging. Computational methodologies to understand, predict and diagnose the different diseases using these data will be a boon to the field. We have devised a methodology to utilize the published data in statistical and graphical (stat-graph) approaches that will uncover deeper insights into the disease-causing mechanisms of these mutations.

The idea is that knowing a published mutation in an ncRNA gene by itself can only show the involvement of this particular mutation in the particular ncRNA gene in causing a disease. However, GDMAP distributes many mutations in the same gene on the gene structure and sequence, wherein the mutations on the different genetic elements are statistically and graphically depicted, where they are drawn to scale on the length of the gene. The frequencies of the mutations on the different elements and their individual bases will reveal key biological and clinical significance of these positions within the different genetic elements.

In addition, we can build PWMs for the allowed sequence variations of the different genetic elements within every type of ncRNA by using the sequences of a large number of ncRNA genes that occur in the human genome. We expect the frequencies of the disease-causing mutations within the ncRNA genes to closely reflect the PWMs of the different elements of the different ncRNA genes. The unique algorithms that we have developed within GDMAP based on the variable frequencies of the nucleotide changes to the other three nucleotides at every sequence position of a genetic element, obtained from published mutations, enables the determination of deleteriousness and disease causality of every mutation from one base to one of the other three bases. Based on these frequencies, the algorithms assign different disease causality scores to the specific mutations at the different sequence positions of every genetic element.

Provided herein is a method for designing algorithms to determine disease causality of mutations in genetic elements based on the differential base changes at different sequence positions of the genetic element.
1. Step 1: Determine scores for all mutations at a particular sequence position, based on the total mutations at a particular position divided by the total mutations at all sequence positions within the element
   a. Determine the total frequency of all of the base changes at all of the positions of the genetic element.
   b. Determine the frequency of all mutations at a particular position.
   c. Determine the frequency score for all mutations at a particular position, by dividing the frequency of all mutations at a particular position by the total number of all base changes.
2. Step 2: Determine the score for a particular mutation at a particular sequence position, based on the specific change of a base into any one of the other three bases at that position
   a. Determine the frequency score for a particular mutation at a particular position, by dividing the frequency of the particular base change by the total number of all base changes at that position.
   b. Similarly, determine the frequency scores of each of the base changes at each of the positions of the genetic element.
   c. Normalize this score of each of the positions based on the highest scoring position.
   d. These normalized scores will represent the disease causality scores of different mutations at each of the positions of the genetic element.
   e. Thus, the disease causality scores of mutations for each base change at each of the positions of the genetic elements are determined.
   f. This algorithm thus enables us to obtain the disease-causality score for a particular mutation occurring in an individual.

### Determining the Disease-Causality Score for Disease-Causing Mutations in Genetic Elements

Defining a score for disease causality based on the statistical graphing of disease-causing mutations from publications can be achieved using an embodiment of the system, as described below:

### Method

1. Determine a score for a mutation at a particular position within the sequence of the element (donor)
   a. Total number of mutations at each position
   b. Total number of mutations in all positions
   c. Score for a position=(a/b)×100
   d. Normalize this score based on the highest scoring position
   e. Example: position 4 will be the highest. Make that number as 100 and calculate the score for each of the other positions
   f. Total number of mutations in all 9 positions=640
   g. Total number of mutations at 4th position=228
   h. Deleteriousness score of 4th position=228/640=0.36×100=36
   i. Deleteriousness score of 1st position=4/640=0.00625×100=0.6
   j. Deleteriousness score of 3rd position=106/640=0.166×100=16.6
2. Determine the score for a particular mutation at a particular position
3. For instance, at 4th position, if the mutation is G→A, then its score is=137/(137+30+60+1)=137/228=0.60×100=60
4. Score=36×60=2160/100=21.6
5. At 1st position=¼=0.25×100=25
6. Score=0.6×25=15/100=0.15
7. At 3rd position G→A=60/106=0.57×100=57
8. Score=16.6×57=946.2/100=9.46

### Normalizing

9. Take the score of the highest scoring position—i.e., 4th position to be 100
10. The score of the 1st position mutating C→A=(0.15/21.6)×100=0.69
11. The score of the 3rd position mutating C→A=(9.46/21.6)×100=43.8
12. Thus, the disease causality score for the 4th position G→A mutation is 100
13. The disease causality score for the 1st position C→T mutation is 0.69
14. The disease causality score for the 3rd position G→A mutation is 43.8
15. In position 1, calculate the number of mutations at position 1

The above described methods and algorithms, and their variations, will be used for every genetic element in protein-coding and non-coding RNA genes.

### Pathogenic Mutations Versus Strength Altering Mutations Revealed in Statistical Graphing Method

Pathogenic mutations in a gene will disrupt the protein to the extent that the protein will lose its structure whereby its function will be lost. The biochemical or biological reaction or process that the protein is involved in will be disturbed to the extent that it can lead to a disease. Strength altering mutations, on the other hand, will not destroy the structure or the function of the protein, but will enhance or decrease the function of the protein, or its production. We predict that this type of mutation can occur in any of the genetic elements such as the promoters, splice donor, acceptor, branch, exon or intron splice enhancers and silencers, poly-A sites and signals, Kozak sequence, and enhancers and silencers of promoters and poly-A signals, or within the coding sequences of genes.

These mutations can either strengthen or weaken these genetic elements in their biochemical or biological activity such as their binding strength to the target molecule, thereby enhancing or reducing the outcome of the process. For instance, a promoter strengthening mutation can enhance the production of the protein thus overexpressing the protein. A splice donor mutation can weaken the donor binding strength to the spliceosome machinery thus reducing the splicing reaction, leading to a reduction in the quantity of the spliced transcript per unit time. An amino acid mutation in the binding site of an enzyme can reduce its binding to its target biochemical such as a cofactor or coenzyme, and thus reduce the kinetics of the biochemical reaction. It may also over-activate or reduce an active site of an enzyme thus altering the kinetics of its biochemical reaction.

Mutations that are not pathogenic or strength altering are categorized as variants of unknown significance (VUS) in the clinical genomics field, as they apparently have clinical significance. We predict that many of the VUS are strength altering or pathogenic mutations, not only in the CDS regions, but also in any of the genetic elements within a gene. When we overlay mutations from publications on the genes, genetic elements, or protein sequences with not only pathogenic mutations but also other mutations by the statistical graphic method, these types of VUS and strength altering mutations will be revealed by virtue of their statistical significance.

### Genetic Elements that Occur at Variable Distances from Fixed Elements in a Gene Revealed by Statistical Graphing Method

There are some elements that occur at relatively fixed positions within a gene, such as the transcription start site (TSS), and donor and acceptor splice sites. However, other elements such as the promoter sites (e.g., TATA box or GC box), cryptic splice sites, poly-A sites, and their enhancers and silencers occur at variable positions with respect to what is generally considered to be the fixed positions of the TSS or the splice donor or acceptor sites. Thus, the mutations on these variable positions cannot be overlaid exactly. In addition, these “movable” genetic elements are known only for a subset of genes. Presented herein is a method to identify these movable elements based on the statistical graphing of a large number of published mutations in and around a gene.

The distribution of the mutations on a statistical graph would reveal a sequence motif that exhibits a high frequency of the mutations with structural or functional significance. This sequence motif can be scored for its resemblance to one or more known genetic elements to identify which genetic element it represents. From thereon, we can use the algorithms we have designed to determine the disease causality of mutations from an individual.

The statistical graphing method would reveal the sequences of the motifs, and the relative weights of the different sequence positions within the motif in this type of genetic elements that occur at variable distances from the fixed elements within a gene. This method will also reveal many unknown motifs throughout the genome that have both biological and clinical significance. These motifs and their mutational nuances revealed by the statgraph methodology will be applicable in clinical setting to diagnose and treat patients with the most effective drugs with least side effects, as they will also be applicable for not only disease causing genes but also for therapeutic genes and drug metabolizing genes.

### GDMAP and Cohort Analysis

The majority of the positions and sequences of the enhancers and silencers of the regulatory and splicing elements of each of the 20,000 genes are unknown. These are known only for a small set of genes. The frequency patterns of a large number of published pathogenic mutations from a large number of patients with a particular disease will show a characteristically higher frequency at these locations, and at each nucleotide position of each regulatory and splicing element within every gene involved in the disease. This is true with genes involved in a drug response phenotype such as effective therapy or harmful side effects.

In addition, the enhancers and silencers of regulatory and splicing elements do not occur at fixed positions. As the promoters, enhancers and silencers of regulatory elements occur at variable distances from the transcription start sites (TSSs) or transcription termination sites (TTS), the GDMAP has the ability to uncover them. The same will be true for the enhancers and silencers of splicing, which occur at variable distances relative to the donors and acceptors of exons. This is true with ncRNA genes.

The genomic study of a disease cohort is expected to reveal the genes and mutations causal of a disease (or any phenotype including drug response phenotypes, or traits such as skin color, height, or longevity of an individual), by bringing out the most frequently mutated genes across the cohort exhibiting a particular phenotype. However, the cohort studies will only show the most frequently mutated genes regardless of the other disease genes present in the cohort due to the other common diseases (such as diabetes or hypertension) exhibited by the members of the cohort. The cohort study will thus erroneously bring up genes causal of other diseases. In contrast, most of the published mutations are expected to indicate pathogenic mutations specific for a disease, as they are expected to have been isolated or purified to indicate the disease of interest. Thus, GDMAP will reveal genes that are specifically causal of a particular disease or phenotype. GDMAP has a module to compare the genes and mutations in the coding, regulatory and splicing elements from a cohort study with those obtained from published mutations for a particular disease.

In summary, GDMAP enables deeper genomic insights by the collective analysis of published mutations of all the genetic elements in the genes causing various genetic and protein aberrations leading to various diseases or phenotypes in a combination of statistical and graphical approaches. This sort of deeper insight is not possible by the analysis of single mutations in individual genetic elements or genes. The approach enables this analysis from thousands of published mutation data, the majority of which are known to cause disease. The derived knowledge from this analysis leads to the understanding of the pathology of a gene that in turn indicates its biology, depicting where in the genome and disease biology the particular gene participates. The understanding of the pathology of mutations in a genetic element through the collective statistical and graphical approach will indicate the genetic and biological environment in which the element within the gene carries out its function. The approach to connect the biology and pathology of the individual genetic elements is able to uncover deeper insights into the molecular causation of the disease.

### SpliceCode Algorithm

Identifying the genetic elements including the coding, regulatory, and splicing elements of a gene, and the complete gene in a raw DNA sequence by SpliceCode algorithm and AI systems

The splicing machinery called the spliceosome employs a molecular and cellular algorithm to identify the regulatory, splicing and coding elements of a gene accurately from a genome sequence. This molecular algorithm inherent to the splicing machinery is termed as the Splice Code. We have understood that numerous pseudo or cryptic regulatory and genetic elements occur throughout the genome sequence including the genic regions. Thus the Splice Code is expected to accurately distinguish between the genuine elements of gene regulation and splicing, avoiding the numerous pseudo or cryptic elements that are strewn around the genuine elements throughout the genes and the genome.

As the cryptic elements highly resemble genuine elements, it has been difficult to understand how the Splice Code is able to distinguish these correctly. Thus far, the SpliceCode has not been deciphered to any extent. We have developed a SpliceCode™ algorithm that closely mimics the cellular Splice Code. This algorithm is able to correctly identify ˜90% of the genes, including all of its genetic and coding elements.

True splice sites have certain sequence characteristics within and around the exons that enable the true exons recognizable as a signal above a threshold by the combination of these sequence characteristics. In addition, underlying themes have been elucidated for the spliceosome in being able to select correct consecutive exons towards a molecular goal. Thus, the goal is to achieve a contiguous coding sequence of the gene, without interruption by any stop codons. In choosing the next exon, the goal of the spliceosome is to find the exon with all of the signals that are characteristic of an exon, plus the continuity of the ORF within the spliced exons thus far in its pursuit of finding the consecutive exons for the gene.

The spliceosome acts on a primary RNA transcript. Thus, the spliceosome's splice code will start from the start of the transcript, to identify the first exon with certain specific characteristics of the first exon. The Splice Code algorithm for defining the first exon is: 1) it should have an initiator codon downstream of the start of the transcript, 2) then it should scan the downstream sequence for the occurrence of the first donor sequence with any stop codon interruption, 3) if there is a stop codon before the donor sequence, then it should start with the next initiator codon, looking for the first donor without any interrupting stop codons. This rule may include the requirement of an exon or intron splice enhancer and silencer within the first exon or intron, and also the presence of Kozak sequence surrounding the initiator codon.

The Splice Code algorithm for defining the second and subsequent middle exons: 1) a middle exon should start with an acceptor signal and end with a donor signal, 2) it should have a continuous coding sequence without interruption by any stop codons on the same reading-frame as that of the 1st exon, and 3) it should have a branch point signal upstream of the acceptor signal. This rule may include the requirement of an exon or intron splice enhancer and silencer within the middle exon or neighboring introns.

The Splice Code algorithm for defining the last exon: 1) it should start with an acceptor signal and end with a stop codon, 2) it should have a continuous coding sequence without interruption by any stop codons on the same reading-frame as that of the combined previous exons, and 3) it should have a branch point signal upstream of the acceptor signal. This rule may include the requirement of an exon or intron splice enhancer and silencer within the last exon or the upstream intron.

An additional rule for all exons may involve protein domains. The contiguity or combination of two exons should correspond with the contiguity of the amino acid sequence of a protein domain, when the domain is coded by the contiguity of multiple exons. If a contiguity is broken and misses a portion of the domain, it would indicate that there is a missing exon at that location.

The cellular Splice Code will be able to identify the first, middle and last exons present in a gene's primary RNA transcript. These exons within the RNA transcript are identified based on the specific features of genetic elements, and the requirement for the contiguity of the coding sequence leading to a contiguous amino acid sequence of the protein, encoded by the gene.

The SpliceCode algorithm identifies the genetic elements responsible for splicing the exons together, such as the donor, acceptor, branch points, enhancers, and silencers. It determines these genetic elements by using their PWMs and similarity scores calculated from algorithms such as the Shapiro & Senapathy algorithm, MaxEntScan algorithm and NNSplice algorithm, their modifications and combinations, or other scoring algorithms. It sequentially identifies the protein coding exons by identifying the first exon, consecutive middle exons, and the last exon that codes for the contiguous protein sequence encoded by the gene.

We have also observed that the length of the genetic elements such as donor and acceptor can be altered or tweaked to identify the genuine elements which improved the scores of the genuine elements in genes. These improved algorithms based on the altered sequences and lengths can also be incorporated into the Splice Code algorithm.

In order for the genetic regulatory system to identify the start of a gene and end of a gene, there are two additional systems. The start of the gene should consist of promoter elements, the sequences of enhancers and silencers present at multiple sites for multiple binding proteins (transcription factors), and a transcription start site at which the transcription of the primary RNA transcript starts. These sequences are fairly unique and recognizable by corresponding PWMs, and we will use scoring algorithms such as the Shapiro & Senapathy algorithm, MaxEntScan algorithm and NNSplice algorithm, their modifications and combinations, to predict these elements.

The end of the gene is a transcription termination site with specific recognition sequences in the gene sequence. These specific recognition sequences occur close to the polyA addition site and signal. There is an additional sequence called Kozak sequence that surrounds the initiator codon ATG and aids in its recognition in the mRNA by the ribosomes. Thus, the SpliceCode™ system will be able to identify the complete set of elements that constitute a gene from a genomic sequence.

### Automated System for SpliceCode

An AI/ML system is described herein, trained to correctly identify the regulatory, splicing and coding elements of a gene using the characteristics of known genetic elements based on similarity scores and other parameters of the genuine elements. Thus, the AI system is trained with the SpliceCode algorithm that we have developed, namely the rules and steps that we have built into the algorithm. The AI system learns the nuances of the rules as applied to the genetic elements, scores, sequences, their positions within the genes, for a large set of genes that we used to train the SpliceCode algorithm. Thereby, the AI SpliceCode system is able to predict the elements of new genes that are given to the system for testing its accuracy.

The AI/ML system is trained to learn the first exons, middle and last exons separately using the S&S and other scores of the elements surrounding each, and certain parameters of these exons (using the requirement for an ORF that encompases the exon, etc), and the introns. As there are >200,000 exons with an average of 10 exons per gene, and ˜20,000 genes in the human genome, a large set of these exons and genes can be used as training and test data sets.

In the above training, in addition to exons, regulatory elements of transcription, splicing, and translation such as the promoter elements, Transcription Start Sites, and poly-A addition elements, Transcription Termination Sites, are provided to the AI/ML system. Next, the ML system is trained to recognize the complete gene parameters of first exons, middle exons and last exons to recognize the complete gene patterns.

When the ML system is fully trained and tested, it can predict complete genes from a genomic sequence. We have developed a software code implementing the SpliceCode algorithm. The outcome of this SpliceCode program can be matched with the SpliceCodeAI™ system to verify the validity of the ML system.

### Method for Rapid Whole Genome Interpretation (rWGI)™

We have devised a method to interpret a patient's whole genome sequence based on the known features and properties of the coding, regulatory and splicing elements of genes within the human genome. We use the known characteristics of their different elements within the gene and the genetic and biological environment in which these elements are dispersed across the gene. The information of all of these structural and functional features are embedded within the gene sequence and can be unearthed by using algorithms that we have developed. The method exploits the similarity scores of the various genetic elements embedded within a gene and the knowledge of the cryptic elements that surround them.

The mutations and the genetic and protein aberrations that they cause leading to various diseases, traits and drug response phenotypes are finite although large in number, and are determinable by using the several algorithms that we have developed. If we are able to determine and identify all possible mutations that lead to deleterious defects in all of the genes and proteins from the human genome, then we can use this set of knowledge to predict all of the genetic and protein effects of a new mutation from a patient that leads to a disease, trait or drug response phenotype.

The method employs the similarity scores of all of the genetic elements that occur in all of the genes calculated using the Shapiro-Senapathy algorithm. The mutational spectrum of the different sequence positions of a particular genetic element that occur in a particular position within a gene can be calculated to obtain all possible mutations in that genetic element. This is applied for every genetic element throughout the gene, and the cryptic elements surrounding it. For example, all possible mutations that occur in the true and cryptic splicing elements that are dispersed within a particular exon and its surrounding introns can be determined, by changing the particular base that occurs at a particular position to other three bases. Some of these variations may be normal variations that have no aberrational effect. However, others may cause molecular aberrations leading to a genetic and protein defect.

The molecular effects and aberrations of these limited number of variations that can occur within a genetic element can thus be calculated. Extending this to all of the genetic elements and their cryptic versions within a gene, and scaling this up to all of the genes within the human genome will enable us to obtain all possible mutations that cause deleterious defects leading to various diseases and other phenotypes. This is also true for the mutations that occur in the coding regions of genes.

The approach of the algorithm is to obtain this large set of all possible mutations and their molecular effects from the whole genome at one time. The algorithm then uses this information to predict the effect of a mutation from a patient, as we have determined that any new mutation or variant from any patient with any disease or phenotype will be among the set of all possible mutations that we have already mapped out.

The algorithm deals with a practical issue that arises when we compare a very large number of variants that occur in the genome of an individual. We have determined that approximately 5 million to 10 million variants occur in the whole genome of every individual. It takes a substantial amount of time to compare this large number of variants with all possible variants from all of the genes in the human genome. Thus, we have overcome this problem by parallel processing different smaller segments of the 5 to 10 million variants to identify the particular positions within the genetic regulatory, splicing and coding elements of various genes.

These positions within particular genetic elements within particular genes will indicate if there are any aberrations caused by that genetic element mutation. There is yet another problem that we deal with in this process. The molecular aberrations caused by two or more mutations that occur within the same genetic element or within the genetic element and one or more cryptic elements surrounding the genetic element have to be determined. These situations are isolated from the whole genome and are parallely processed to obtain the pathogenic or deleterious mutations that have the potential to lead to disease.

The predictions based on the calculations and experimental observations in carrying out this process using the variants from the whole genome of an individual are the following (numbers are approximate estimates):
1. The total length of the sequence across all 20,000 genes is ˜1.2 billion bases (genic regions).
2. The total number of all possible variants in the genic regions, obtained by mutating each base to other three bases, is ˜3.6 billion bases (1.2 billion×3).
3. The set of pathogenic mutations (lookup) for all possible mutations in all genetic elements in all ˜20,000 genes, calculated from similarity scores, is expected to be ˜20-40 million.
4. The number of variants from an individual's whole genome sequence is ˜5-10 million
5. This set of variants from an individual's whole genome sequence will have ˜300,000 pathogenic mutations in all of the genetic elements of all genes.
6. We estimated approximately 25,000 genetic elements, in which a pathogenic variant occurs, will have one or more additional variants within the same or neighboring genetic elements.
7. The set of pathogenic variants with aberrations (molecular effect) is estimated to be ˜2,500 indicating that one in approximately 100 pathogenic variants will lead to a molecular aberration.
8. We will segment the total number of pathogenic genetic element mutations in the genome and the total number of variants from the individual into multiple segments, and process them parallely to obtain the pathogenic genetic element mutations in the individual.

Devised is a methodology to identify all possible pathogenic mutations in the CDS regions of all ˜20,000 genes. This is achieved by subjecting the coding sequence to the predicting software for pathogenic mutations in the CDS regions (using the algorithm called Comprehensive Variant Classification, CVC).
1. The total length of the coding sequence across all 20,000 genes is ˜62.5 million bases (coding regions).
2. The total number of all possible pathogenic mutations in the CDS regions is estimated to be 5-10 million.
3. This set of pathogenic mutations will be used as a lookup to compare the ˜5-10 million variants from an individual, to obtain the CDS pathogenic mutations and the genes they occur in an individual.
4. We will segment the total number of pathogenic CDS mutations in the genome and the total number of variants from the individual into multiple segments, and process them parallely to obtain the pathogenic CDS mutations in the individual.

The rWGI system enables the processing of all of these steps in a relatively very short time. This algorithm thus enables us to accurately interpret all of the 5 to 10 million variants in the complete genome of an individual within a very short time.

### Rules

1. Mutation in a real splice site→looking for cryptic splice site→check if that cryptic site has any variants in that patient→consider the patient variant→effect calculation for patient variant
   a. Generate a master lookup that has all possible pathogenic mutations in all RSE (regulatory and splice elements) elements (real+cryptic) throughout the genic region (1.2 billion bases)—based on % difference only→name it as ‘master lookup’ (˜50 M)
   b. Generate an effect lookup that has all possible pathogenic mutations in all RSE elements (real+cryptic) throughout the genic region (1.2 billion bases)—based on effects→name it as effect lookup
   c. Match the patient data (˜5 million variants) with the master lookup→you will get all pathogenic mutations in a patient based on % difference→this will be around 300,000 for ˜5 million variants
   d. Parse these 300,000 mutations
      i.Find if the input patient data has any variant surrounding the mutation position (+/−300 from the mutation position)
         1. For instance, if there is a P mutation at position 1,000 in chr 1—gene:TP53, exon 3, real donor, then find if there are any variants within 700 to 1300
      ii.If you find a variant, incorporate that variant to the refseq.
         1. Check if the variant occurs in any cryptic/real site
         a. If yes, calculate its effect from scratch (same as current SA parser does)
         b. If no, proceed to step (iii)
      iii.If you do not find a variant from the patient file within the specific range, then match that particular mutation with the effect lookup to find its effect
   e. These 300,000 mutations can be processed as chunks of 3000 variants each and processed parallelly on 100 instances→time will reduce 100 fold
2. Also check for more than one mutations in one element

### AI/ML System for rWGI

The most limiting factor in whole genome interpretation is the longer turnaround time. To overcome this limitation, we have developed a technology called rapid whole genome insights. Through this technique, the whole genome sequence date can be interpreted in less than a few hours. By applying AI/ML techniques, the interpretation time can be further reduced. From the whole genome reference sequence, all possible variants in every genetic element such as an exon, acceptor, donor, branchpoint, promoter sites, polyA, enhancers and silencers, and introns, were generated. S&S and other algorithms are applied to these variants, and their features such as sequence, variant score, and neighboring elements are determined to determine the pathogenicity and disease causality of the variants that have the potential to cause disease or drug response phenotype.

The AI/ML systems are trained with these known features of all possible pathogenic variants in the reference whole genome. The trained systems will be able to identify pathogenic disease or actionable mutations from the list of all possible variants from the whole genome. These disease causing and actionable pathogenic variants, and their characteristic features are used as a lookup to interpret a patient's genome data, which enables a fast turnaround time for interpreting a patient's genome.

### Genome Artificial Intelligence™—GenomeAI™

It is estimated that there are approximately 5 million variants from the whole genome sequence of every individual or patient, compared to the reference sequence (Eg., Ensembl, RefSeq). However, only a miniscule fraction of these variants have deleterious effects on gene regulation or splicing, or the protein itself. Moreover, on average, it is estimated that ˜1,000 deleterious mutations occur within every individual's genome. Additionally, the possible number of deleterious mutations in a given gene is finite, and thus by one embodiment of the presently described system to categorize deleterious mutations, it is possible to discover and identify novel mutations not previously described in literature.

The aim is to develop algorithms and methodologies that are capable of distinguishing and identifying deleterious mutations from the non-deleterious variants from the totality of 5 million variants of a patient. In addition, the aim is to collect one or more known deleterious mutations from one or more known genes from published literature that cause any disease or drug response phenotype, along with other relevant details. According to one embodiment of the present system, the output will be a complete dictionary and encyclopedia of deleterious mutations from genes in the human genome that cause human disease and drug response phenotype, from which one can simply lookup one or more variants of a patient to see if it has a deleterious effect and if it would cause a disease, and what disease. Thus, the system will allow us to identify the very small miniscule of disease and drug response causing deleterious and strength altering mutations from nearly 10 billion possible variants in the human population.

Only 1 in ˜1000 variants that occur in an individual is a pathogenic/deleterious mutation that has any effect on gene expression, splicing or protein sequence, and are causal of disease or drug response phenotype. We aim to use the algorithms, methodologies and technologies, capable of identifying deleterious mutations in gene regulation and splicing, and protein structure and function, along with validated deleterious mutations, and overlay and train AI/ML technologies to be able to predict novel deleterious mutations in genes and proteins.

Thus, one embodiment herein will systematically accumulate details and data relating to the major categories of biological features or processes, namely, gene expression, splicing, translation that cause aberrations in the protein (exonic) sequence of each of the 20,000 genes. We will sub-categorize each of these major categories and approach them systematically to build the evidence based algorithms and technologies. Next a machine learning system will be trained on the validated data.

Three different possible types of deleterious mutations can occur within a gene and lead to disease:
1. Gene expression regulation—promoters (TATA, CAAT, GC, initiator elements), polyA site or signal, their enhancers or silencers can affect transcription and transcript processing, translation regulation (Kozak and other sequences, microRNAs and other regulatory elements).
   a. They affect the level of gene expression.
2. Splicing regulation—acceptors, donors, branch points, splicing enhancers and silencers.
   a. They affect the processing of splicing to bring together exons and delete the introns of a gene. It can cause large deletions of a protein sequence or insertions of intronic sequences into the protein sequence, thus greatly affecting the actual amino acid sequence of the protein.
3. Deleterious mutations within the actual amino acid sequence—coding sequence mutation.
   a. These are SNPs and INDELs that occur within the coding sequence (exons) of the gene, and deleteriously affect the protein structure and function.

The following steps can be taken to identify the training datasets and then train an AI/ML system to predict the real donors, real acceptors, and real exons:
1. First train real donors alone in 1000 genes separately
   a. Take real donors as defined in the gene annotation for 1,000 genes
      i.Obtain their Shapiro-Senapathy (S&S) similarity scores by subjecting the donor sequences to the algorithm
   b. Train an ML system with
      i.Only the donor sequence and their position in the gene sequence
      ii.Next, use S&S score in addition to the donor sequence and position in the gene sequence
      iii.Next, subdivide the donors based on their score ranges, such as the scores of 90-100, 80-90, . . . <50, etc.
      iv.Train with the subsets of donors within each score category, by specifying their positions within the gene, their sequence, score
      v.Next, specify in addition to the features in (iv), other parameters and features one by one, and so on, such as acceptors for the exons containing the donors being analyzed, their scores, sequence, etc., branch points, and so on.
2. Next, train real acceptors alone in 1000 genes separately, following the Step 1 above.
3. Similarly, train the real exons alone by providing the exon sequence, position, S&S score of exons from 1000 known genes to the AI/ML system, adding each in successive steps
   a. In this step also, as in step 1, obtain the S&S scores for exons, and categorize them into subsets, such as 90-100, 80-90, etc.
   b. Train the ML system with the different categories of exons
4. Then, train the first exons of genes alone. In this step, give requirement for the elements that constitute the first exon, including the Kozak, ORF from Kozak start codon to the end of first exon, in addition to ending in a donor splice site, and the requirement of an ORF between the start codon to the occurrence of the first donor.
   a. In this step also employ the nuances as in step 1 and step 3.
5. Similarly, train the last exons alone from 1,000 genes, incorporating the requirements genetic elements that constitute a last exon, including the Poly-A addition signal and site, enhancers and silencers of transcription termination and poly-adenylation.
   a. In this step also employ the nuances as in step 1 and step 3.
6. Next, train the middle exons in a similar manner. The examples of its requirements are acceptor to donor 30 to 600 bases length, ORF in at least one of the reading frames, RNY periodicity in the same reading frame as that of the ORF that matches with the exon, exon score thresholds for donor, acceptor, branch point, exon splice enhancers (ESE), intron splice enhancer (ISE), exon splice silencer (ESS) and intron splice silencer (ISS).
   a. In this step also employ the nuances as in step 1 and step 3.
7. Similarly, train the AI/ML system with details of a combination of real exons (only the sequence) and real donors
   a. Add the S&S scores for donors and exons
8. Next, train with the combination of real exons (only the sequence) and real acceptors
9. Train the system with details of combination of real exons, real donors, and real acceptors, with respective sequences including or excluding the S&S score
10. Train the AI/ML system with known RNY periodicity in exons of multiple genes (e.g., 1,000 genes).
11. In addition, the system is trained with other elements added one by one (Branch, ESE, ESS, ISE, ISS, etc).
12. Continue training on this line with each element with sequence including or excluding S&S score
13. In training and testing exons:
   a. Include one exon in one or more genes
   b. . . . +Its neighboring 300 bases on the right
   c. . . . +Its neighboring 300 bases on the left
   d. . . . +Its neighboring introns
   e. . . . +its neighboring introns+exons
14. Next, train with a complete gene, with one or more of the elements successively included by combining the genetic elements one by one, for example, promoter+first exon, promoter+first exon+second exon, promoter+first exon+middle exons+last exons, then add poly A site, etc.
15. Once the system is trained thoroughly with individual elements, various combination of the elements and the whole gene, it can be tested with an untrained set of 1000s of elements and genes for each step of training. The training and testing datasets will be divided and chosen randomly to allow for a large number of unbiased datasets. The trained and tested system should be able to predict one or more elements, exons and genes.

The gene sequence contains not only the real genetic elements where they are expected to occur, but also numerous spurious or cryptic sequences that resemble the real elements that occur throught the gene and the genome. Another way to make sure that the system will identify the real elements specifically, as described above, is to mix cryptic elements resembling one or more types of real elements during training and testing. When training with complete genes, the cryptic elements occur throughout the gene, and therefore it provides the environment wherein the real elements and cryptic elements are interspersed.

Training the AI/ML system with real and cryptic elements in a systematic manner as described above, and the exons and the complete gene, would enable the trained system to predict a gene from a sequence with only its real genetic features, thus avoiding the cryptic features, and the vice versa. The ability has important implications in identifying the genetic mutations in a gene capable of causing a disease or drug response.

The following steps describe the steps to identify the training data sets, and then train an AI/ML system to identify deleterious mutations in the different elements or features of a gene.
1. Train and test the ML system with known deleterious mutations that are known to cause a disease in individual genetic elements such as a donor splice site first
   a. In this step, subdivide and categorize the splice sites based on several parameters such as the Shapiro-Senapathy score, as described above in the previous section (on predicting real donors, etc.), and follow the other nuances as well.
2. In the next step, mix benign (i.e., non-deleterious) mutations along with deleterious mutations
3. Next, train and test successively by adding first exons, middle exons and last exons containing deleterious mutations, and then non-deleterious mutations at various sequence positions. Additionally, train with the nuances as described above.
4. Conduct this line of training and testing with known, disease-causing mutations in the different genetic elements and features of a gene in a systematic manner as described in the steps above
   a. For example, do the training for multiple elements of an exon with mutations in different places of an exon, and train on 1000 exons
   b. Similarly, do the training for mutations within introns with its multiple requirements to identify near and deep intronic mutations that are known to cause disease
   c. Next, train the system with disease causing mutations in complete genes
   d. In these steps, give only real exons in a gene for training, and then cryptic exons
   e. Give requirement for ORF continuity from start codon of first exon to the stop codon of last exon, and train mutations that affect this continuity in the mRNA sequence
   f. Then add requirement for S&S score thresholds for each element and the mutated version of the element
      i.Next, categorize the donor mutations into the type of effects that they cause, such as exon skipping, intron inclusion, cryptic exon creation, etc.

Train the AI/ML system with scoring algorithms to identify the aberrational effect of splicing, with mutations in every type of splicing element. Use a set of 1,000 mutations for each type and their effects that are validated as the training and test data set.

For example, when a real donor is mutated:
1. Check if the S&S score percentage difference before and after mutation is in a range of score threshold (e.g., =>3 or <=−3)
2. Search for cryptic donors from a few bases within the exon (e.g., 31st base) to a length range within the intron (e.g., 1st 300 bases) with respect to mutation position.
3. Check if the S&S score of the mutated donor is lesser than the cryptic donors
4. Calculate the distance of the cryptic donors within the mentioned range from the real site
5. If the minimum distant cryptic donor lies in the intron, then the effect is intron inclusion
6. Similarly, if the minimum distant cryptic donor lies in the exon, then the effect is partial exon deletion

Similarly, based on the specific nuances of each of the genetic elements such as acceptors, branch points, enhancers, or silencers, appropriate aberrational effects are determined.

In the same manner, continue with mutations within one or more promoter elements such as the TATA, CAAT, GC boxes, and one or more poly-A addition elements, separately and in various combinations thereof. In this, categorize the mutations into the type of effects that they cause, such as increase in transcription, decrease in transcription, or abolition of transcription, and various nuances in between

Train one or more enhancers or silencers of gene expression (gene regulators) for a gene like TP53 with mutations known to be deleterious or non-deleterious. Some of these enhancers or silencers occur in multiple genes—i.e., the same target enhancers or silencers occur in multiple genes, and are bound by the same Transcription Factors. In this process, we can use the known consensus sequence(s) for a gene like TP53 binding DNA sequences that occur in many genes. When trained with one or more such genes in the genome that have the same target Transcription Factor binding sequences, it is possible to identify the genetic effect of the mutation causal of a particular molecular and disease causation in regulatory genes such as the TF genes and their target binding DNA sequences. Similarly, do this for mutations in different enhancers of multiple genes containing the same TF binding sites.

The corollary of the above described process—i.e., many different silencers and enhancers occur surrounding one gene, for example, the gene TP53. Each of these silencers and enhancers are targeted by a different Transcription Factor gene. The ML system will be trained with thousands of genes with mutations in the same or different enhancer or silencer. As in other training and testing data sets, random datasets will be used here also. The trained and tested ML system would be able to identify one or more disease or drug response phenotypes based on a mutation in one or more enhancers or silencers in one or more genes in the genome.

In all of the above training, use mutations in different genetic elements including splice sites, exons, promoters, poly-adenylation sites, enhancers and silencers, that are known to cause molecular effects such as enhancement or suppression of transcription, splicing errors such as whole or partial exon skipping, partial intron inclusion, cryptic exon creation, premature termination codon creation, enhancement or suppression of poly-adenylation or translation initiation, as the training datasets, and train each of these molecular effects.

In these training, the system will further use random mutations simulated in one or more positions of the genetic elements, exons, and genes. The system is also configured to use similarity scores calculated by algorithms such as the Shapiro-Senapathy, MaxEntScan, NNSplice and their modified versions thereof. Algorithms such as Shapiro-Senapathy are capable of predicting the consequence and effects of splicing mutations in disease causality. We will use the available data in the training and testing.

Deep intronic mutations are now known to cause approximately 50% of all diseases. We will train with deep intronic cryptic genetic elements (i.e., ˜300 bases into the intron on both sides of the exon) to detect them. We will then use known mutations in them to train, test and identify molecular and disease causality. In addition, we will use algorithms like S&S to predict deep intronic mutations that cause molecular and disease abnormalities, which data can be used in training and testing.

In another embodiment, artificial intelligence (AI) and machine learning (ML) techniques are applied to the algorithms that are developed by statistical graphing of published mutations in the GDMAP platform. In this embodiment, mutations from publications in genes that are known to cause diseases are assembled, and subdivided into various categories and sub-categories of mutations in different genetic elements of genes, and their parameters such as S&S scores, their molecular effects such as exon-skipping, their statistics in different elements, and so on, and are studied for association with disease causality. Similar approaches are carried out for genes and mutations for therapeutic indications and harmful side effects indications. ML systems are trained systematically in these details and various parameters with training datasets of known features, mutations, and disease and drug responses, and tested with appropriate datasets. The trained and tested ML systems are used in actual clinical setting to identify these mutations causal of disease and drug response phenotypes.

The ML system is trained on the statistical graphing algorithms with the objective of enabling it to predict pathogenic and strength altering mutations in various genetic elements of the genes. We also apply ML techniques in all the modules of Splice Atlas and Genome Explorer platforms. The modules in GDMAP, the maps of Splice Atlas and the modules of Genome Explorer apply the Shapiro-Senapathy algorithm, and other known and novel algorithms, to determine the scores for different genetic elements and predict mutations in them. The S&S scores for the majority of the donor splice sites within genes, for example, can vary substantially between 70 and 100, while a minority exhibits lower scores, even as low as 40. We train the ML module to recognize the sequences of donor, acceptor, and other types of genetic elements with scores within a specific range (e.g., 90-100, 80-90, 70-80, 40-50, or <70), by giving the sequences, positions, and scores of the different splice sites within different genes, and the complete gene sequences. The trained AI system will then be able to predict these splice sites within varying score ranges or strengths in a new set of genes.

The data sets for training are subsets of splice sites such as the donor, based on S&S (or other algorithms) that occur within specified ranges, for example, 90-100, 70-80, <60 etc., that occur within a set of genes. Another subset of splice sites from another subset of genes from the human genome can be used as the test data set. A similar process is carried out for cryptic splice sites (e.g., cryptic donor sites).

Genome Explorer predicts and categorizes mutations in different genetic elements based on algorithms that takes into account the scores of the elements and their mutated forms, and the differences in scores above or below a threshold. This algorithm then categorizes different types of mutations into deleterious, strength altering or non-deleterious mutations. The ML system also is trained and tested to detect these different types of mutations using the known data, and to predict unknown data.

The ML systems can be applied to other genetic regulatory elements such as the promoters or poly-A addition sites. The scores for the various regulatory elements, such as promoter elements, polyA elements, enhancers and silencers of these elements, also vary within different ranges, and the ML system is trained to predict them and their mutations. There are numerous regulatory elements that occur in and around every gene, including the enhancers and silencers of every gene, with specific target binding sequences and their corresponding binding proteins (or RNA molecules such as microRNAs). The ML system is trained to be capable of distinguishing the sequences, their strengths (similarity scores), the positions, and their mutations in different genes.

Cryptic genetic elements that resemble genuine elements occur at numerous positions in the gene. However, the scores of cryptic elements resemble the true elements, and are often higher than the true elements that occur nearby. Thus, it is important to distinguish the true and cryptic elements to be able to predict them correctly. Furthermore, mutations in true elements erroneously lead to the use of higher-scoring cryptic elements that occur nearby. In addition, mutations in cryptic elements make it possible for them to be erroneously selected instead of the true sites. These types of mutations will cause aberrations in the normal regulatory and splicing processes and lead to numerous diseases.

The ML system is trained to predict the true and cryptic elements based on their genetic environment (specific and relative positions within the sequences of other real and cryptic elements in a gene), and the various possible types of mutations and molecular aberrations. The molecular effects of mutations in different types of regulatory and spicing elements also vary, such as overexpression, underexpression, or abolition, or alteration of transcription, splicing or translation, and exon skipping, intron retention, or pseudo or cryptic exon creation. The ML/AI system is trained to predict and distinguish between the various molecular effects with the corresponding datasets. Thus, GDMAP uses the capabilities of ML/AI to train and predict deleterious or strength altering mutations, disease causality, actionable therapeutics, and drugs to avoid due to mutations in one or more genetic elements of genes.

The GDMAP trains the ML system with a sample training set of genetic elements such as the donor in a sample set of genes (e.g., 1000 genes), before and after a pathogenic mutation causing a disease or drug response phenotype is introduced, by providing the sequence positions of the genetic elements within the genes and the complete gene sequence. In addition, it also trains the system with an added data of the similarity scores (e.g., S&S score) of the elements before mutation and after mutation. This trained AI system will be able to identify disease causing pathogenic mutations in the genetic elements in a new set of genes.

The Splice Atlas platform deals with exons, introns, splicing elements, scores of genetic elements, protein domains, proteins, exon skipping, and the effect of exon skipping leading to frameshift and premature termination. GDMAP trains the AI with these data points and predicts the effects of mutations such as exon skipping, intron inclusion, cryptic exon creation, etc., in a new set of genes with mutations. Likewise, the ML system will be trained with appropriate mutation data for the alteration in functionalities of Splice Atlas and Genome Explorer to be able to predict them in genes.

### Training ML Module with Mutations from Publications

Publications in the field of clinical genomics consists of genetic elements including the coding sequence, regulatory elements such as the promoter, TATA box, GC box, CAT box, transcription initiator and terminator, and splicing elements such as the donor, acceptor, branch point sequence, the enhancers and silencers of all of these elements and their cryptic versions thereof. In addition, they contain details such as the mutations that occur within the sequences of these elements, the molecular effects and aberrations on the elements, genes and proteins. Furthermore, they provide detailed information concerning the disease, therapeutic treatment, their outcomes, adverse drug reactions (ADRs), actionable genes and mutations, and the drugs to avoid due to ADRs.

As described herein, presented is an AI/ML (Artificial Intelligence, Machine Learning) system trained to extract useful and necessary information such as genes, mutations, genetic elements, molecular effects, disease, therapeutics and ADRs from different publications. Moreover, the system is configured to provide insight into the unique combinations between these features to be able to correctly identify and isolate the clinically meaningful information. The AI/ML program is initially trained with the correct set of combinations of information from a set of publications, and the added information such as genetic elements, their similarity scores, mutational aberrations, in a learning process. Then, additional mutations or known variants from other publications are compared against as test data to assess efficacy and iteratively improve the model's accuracy. In one embodiment, results that are obtained by non-AI computational algorithms and approaches (without using AI) are then compared with the results from the AI systems to improve the predictive capabilities of each method.

The same procedures can be carried out for training and testing therapeutic genes and mutations. In addition, the same can also be applied for training and testing Adverse Drug Reactions (harmful side effects) causing genes and mutations.

In training with mutations that cause one or more types of splicing aberrations, train with one type of a splicing element, such as donor mutation that cause one particular type of splicing aberrational effect, for example, exon skipping. Next, train with each of the other types of splicing elements. Similarly, use one type of promoter elements such as TATA box, CAAT box, initiator box, poly-adenylation signal and site, which cause one type of aberration such as increasing the gene expression or decreasing it, or its abolition.

In addition, based on the specific nuances of each of the gene expression regulatory elements such as promoters, polyA, enhancers, or silencers, appropriate aberrational gene regulation effects are determined. Based on the specific nuances of each of the gene translational regulatory elements such as Kozak, microRNA, microRNA target sequences, appropriate aberrational effects in gene translation are determined.

We can also train and test all of the above methods only for a panel of genes that are known to be causal of a particular disease or drug response phenotype, and only for individual genes for which data are available. The same can be done separately for therapeutic genes (drug-gene panel) and for ADR genes (PGx panel).

Certain elements such as one or few donors within a gene of 20 exons will be more pathogenic causing a disease or drug response phenotype—due to their sequence or genetic environment—than others in the gene. The ML system can be trained on this set of highly pathogenic elements within a gene, and can be used to predict such nuances of disease causality within a gene.

### AI/ML for Coding Sequence Amino Acid Variability and Mutations

In another embodiment, described herein is a method to define the variable amino acid (AA) sequence signatures of protein domains by assembling the non-redundant amino acids at every sequence position of a domain. As the variability of AAs is context dependent based on the structure and function of a domain, there are hidden features in these signatures that are trainable.

The ML systems will be trained to understand inherent amino acid sequence variability (domain signatures defined by the proprietary algorithm) based on the sequence context and the biochemical and biological function of the domains and proteins. For this purpose, data on their biochemical/biological functions, active site or binding site amino acids, structure constraints such as hydrophobic/hydrophilic data, and other biological details will be associated with the domain details. The described ML system will also understand the co-dependencies and co-occurrences of particular amino acids at different positions within a variable amino acid sequence signature of a domain or a protein. The ML system can also be trained with the inherent invariance and high or low variance of amino acids at the different positions of a protein domain, based on their restricted variability due to their structural and functional constraints.

The proposed ML system therefore uses the following steps for training variable domain acid sequence signatures.

Take only the human domains from PFAM.
1. Retrieve 1000 domains from only SEED sequence domains
2. Train the ML system with the human sequence and the rest of the seed sequences
3. Test 1000 untrained set of domains, with only one invariant sequence
4. The trained ML system should result in the set of variable AA sequences of the test domains
5. Iterate steps 2-4 to get a high rate of success, each time altering the training and test data by randomly choosing the training and test data from a large set of known and validated data.
6. If the results have a high enough accuracy, then the system can identify the domain Variable AA sequences for the human proteins which are not present in the accessed resource (i.e. PFAM).

The above method should be able to find the “orphan domains” that are present in the human proteome which are yet to be discovered, described, or otherwise studied.

Moreover, described below are additional features of a domain AA sequence to be used in a training set for machine learning:
1. Hydrophobic and hydrophilic AAs
2. Size of AAs
3. Nearest neighbor AA frequencies for 2, 3, 4 AAs in domains
4. Domain 3D structure data
5. Domain hydrophobic and hydrophilic structures data
6. Protein secondary structures data
7. Use Ramachandran phi psi plot to predict the structure of a protein which will help in predicting the nuances of the amino acid variability based on the structural constraints. Train the AI/ML system based on these constraints with known data.
8. The Ramachandran plot is a plot of the torsional angles—phi (φ) and psi (ψ)—of the residues (amino acids) contained in a peptide. In sequence order, φ is the N(i−1),C(i),Ca(i),N(i) torsion angle and ψ is the C(i),Ca(i),N(i),C(i+1) torsion angle.
9. Detect and predict co-dependent or co-occurring AA in a AA variability signature

Thus, described herein is an algorithm wherein the allowed amino acids in a variable amino acid sequence signature are considered to be a positive sequence space. All other amino acids from the set of 20 amino acids, depicted within a grid with X-axis as the sequence position and Y-axis as the 20 AAs, are considered to depict a negative sequence space.

An amino acid within the positive space mutating to another amino acid within the positive space may be classified as non-deleterious. In contrast, an amino acid from a positive space mutating to an amino acid in the negative space may be classified as deleterious, and cause the protein to become defective compared to the non-mutated version. The ML system is first trained with datasets of positive (+ve) space and negative (−ve) spaces for known domains, with the objective of being able to produce variable AAs from −ve AAs given the variable AAs from the +ve space, and vice versa. In addition, given an invariant AA sequence from the variable sequence signature of a protein domain, the ML system will be trained to produce the variable sequence signature for that domain.

In a similar manner, in one embodiment, an AI/ML system is trained with a dataset of CDS (coding sequence) mutations comprising known algorithms and validated data described herein. In some embodiments, the system is trained with mutation detection based on positive negative amino acid variable signatures.

Additionally, the mutational effects of these amino acids in a protein domain, their deleteriousness and disease or drug response causality will also be trainable using known mutations that cause such effects. Thus, the system can leverage the technologies of AI/ML to train and predict disease causality, actionable therapeutics, and drugs to avoid due to mutations in the coding regions of genes.

For this purpose, we can use the CDS mutations that are known to be highly accurate by lab experimentation and in clinical validation. We can use other known parameters and methods that indicates deleterious CDS mutations causal of disease. Finally, we can combine the trained ML system for the regulatory and splicing elements mutations, and the ML system for coding sequence mutations, and this combined ML system should provide highly accurate results of predicting disease-causing mutations in the complete gene, including regulatory, splicing and coding sequences.

### AI/ML for Non-Coding RNA Genes, Their Genetic Elements, Mutations, And Diseases

Non-coding RNA (ncRNA) genes (microRNA, small nuclear RNA (snRNA), small interfering RNA (siRNA), long ncRNA, and snoRNA) are also important factors that cause various diseases when pathogenically mutated. ncRNAs also possess genetic and regulatory elements like protein coding genes, and mutations in any of those genetic and regulatory elements can lead to deleterious effects. Many publications in literature possess data on ncRNA studies and their mutations in various diseases. In one embodiment, an AI system is trained with literature derived sets of ncRNA genes, mutations, positions, genetic elements, similarity scores, sequences, diseases and drug responses from publications. In some embodiments, the system may be trained with different types of microRNAs as described supra, and the regulatory systems of expression and splicing of each of the rnicroRNAs. The trained AI system is then able to predict disease causing mutations in the genetic elements of a new set of ncRNA genes and mutations in new patients.

### Artificial Intelligence and Machine Learning Techniques for Identifying Genome Features and Disease Causing Mutations

Machine Learning (ML) methods may be adopted to train a model to identify features and nuances of the regulatory and splicing elements of genes in the human genome, and the pathogenic mutations that cause disease and drug response phenotypes. These embodiments herein are provided as examples, and are not intended to cover all possible AI/ML (Artificial Intelligence/Machine Learning) methods and procedures.

A Machine Learning approach has been developed to accurately predict the pathogenicity of DNA sequences based on identified regulatory and splice elements. The project's goal was to provide a method for forecasting the pathogenicity of DNA sequences by analysis of known and computer identified features, which may be used as a training data set to train a Machine Learning (ML) model. In one embodiment, the end result is a robust Supervised learning-based Random Forest model that can effectively predict the pathogenicity of new DNA sequences based on their numeric characteristics. This model has the potential to greatly improve the accuracy and efficiency of pathogenicity assessments in the field of genomics.

Described herein is a model trained with a supervised learning technique and a Random Forest model to analyze a training dataset of DNA sequences with known pathogenicity labels. The model is then used to make predictions on new DNA sequences, based on their regulatory and splice element characteristics. In some embodiments, the output can be a binary classification i.e. (pathogenic or non-pathogenic). In some embodiments, the output can be a numerical score representing the degree of pathogenicity. In some embodiments, the numerical score can be 0 (representing non-pathogenicity), or 100, (representing pathogenicity), or any value in between.

### EXAMPLE 2

Described below is a methodology of an embodiment of the AI/ML system described herein.

### Step 1: Curation of a Proper Dataset

A total of 2,28,290 splicing variants (43,843 Real Acceptors, 34,864 Real Donors, 78,018 Cryptic Acceptors, and 71,565 Cryptic Donors) were selected from the ValidSpliceMut database, a repository of validated and cryptic mRNA splicing mutations across various types of cancer.

The process of filtering the collected variants involved categorizing variants based on matching consequence (Real Acceptor/Real Donor & Cryptic Acceptor/Cryptic Donor) as obtained from a data source such as SQUIRL, resulting in a final count of 167,814 variants. Further refinement was accomplished by trimming the variants using the consequence match in S&S algorithms, reducing the variant count down to 94,794. To reassess pathogenicity, a comparison was made between the pathogenicity values from SQUIRL and Shapiro and Senapthy (S&S) algorithm, resulting in a final variant count of 63,993. Only the variants with matching pathogenicity were selected, with a final count of 61,401 (Pathogenic—56,952, Benign—4,489).

### Step 2: Data Transformation

Data Transformation was performed on the data set, transforming the data into a more suitable format for modeling, such as scaling or normalizing using Min-Max Scaler. Missing data was filled in using Data imputation, to replace missing values with estimated values. Dimensionality reduction on the data set was further performed to reduce the number of potential features (i.e. variables) used in the model.

### Dimensionality Reduction Example

#Calculate the Pearson correlation coefficient
between all the features
corr = df.corr(method=‘pearson’)
#Select the upper triangular of the correlation matrix
upper = corr.where(np.triu(np.ones(corr.shape),
k=1).astype(np.bool))
#Find the index of the feature with the highest
correlation to others
to_drop = [column for column in upper.columns
if any(upper[column] > 0.95)]
#Drop the highly correlated features
df = df.drop(df[to_drop], axis=1)

### Step 3: Feature Selection. Selecting the Most Important Features for the Model, by Using Feature Importance Scores

### Dimensionality Reduction Example: Function for Feature Selection Using ANOVA F-Test

```
def select_features(X_train, y_train, X_test):
  # configure to select all features
            fs = SelectKBest(score_func=f_classif, k=20)
        # learn relationship from training data
            fs.fit(X_train, y_train)
        # transform train input data
            X_train_fs = fs.transform(X_train)
        # transform test input data
            X_test_fs = fs.transform(X_test)
            return X_train_fs, X_test_fs, fs
# Feature selection
X_train_fs, X_test_fs, fs =
select_features(X_train, Y_train, X_test)
```

### Step 4: Selection of a Proper ML Model

Supervised, unsupervised, and deep learning are the three main types of machine learning algorithms:
1. Supervised learning is a type of machine learning where the algorithm is trained on a labeled dataset. The goal is to learn a mapping from input variables (features) to an output variable (label). The model makes predictions on unseen data based on the learned mapping. Examples of supervised learning algorithms are Linear Regression, Logistic Regression, Decision Trees, Random Forest, and Support Vector Machines (SVMs).
2. Unsupervised learning is a type of machine learning where the algorithm is trained on an unlabeled dataset. The goal is to identify patterns or structures in the data without any prior knowledge of the labels. Examples of unsupervised learning algorithms are K-means Clustering, Hierarchical Clustering.
3. Deep learning is a subset of machine learning that uses artificial neural networks with multiple layers to complex model patterns in data. It is used for tasks such as image classification, speech recognition, and natural language processing. Examples of deep learning algorithms are Convolutional Neural Networks (CNNs), Recurrent Neural Networks (RNNs), and Long-Short Term Memory (LSTM) Networks.

### Step 5: Training the Model

The Random Forest algorithm is a composite machine learning method that involves building multiple decision trees using the Bootstrap sampling method. This method involves randomly selecting 1000 samples from the raw input data and using them to construct 1000 decision trees. In this algorithm, two types of variables are used, namely dependent variables (Y) and independent variables (X). The independent variables are numerical features that have been calculated, while the dependent variable represents the outcome.

The construction of an RF model, or any other machine learning model, requires the division of the reference datasets into training and test data. In this case, a dataset of 61,401 splicing pathogenic and benign variants was divided into training data (70%, or 42980 variants) and test data (30%, or 18421 variants) using the “train_test_split” function from the scikit-learn (sklearn) library in Python. The RF model was trained using the training data and used to predict the test data. The model was specifically designed to predict the pathogenicity of regulatory splice element variants as either pathogenic or benign.

The RF model was trained using the “RandomForestClassifier” function from the scikit-learn library. The accuracy of the model was evaluated using the metrics module from scikit-learn and further analyzed for improvement.

### Step 6: Testing the Model

There are several evaluation metrics that can be used to assess the performance of the RF model, such as accuracy, precision, recall, F1-score, and AUC (Area Under the Curve) for binary classification problems, and Mean Absolute Error (MAE), Root Mean Squared Error (RMSE), and R-Squared for regression problems. Table 1 illustrates evaluation metrics collected for the 18,421 variants from the Test Data.

TABLE 1
Model Performance on Test Data ( 18,421 variants)

| | Real & Cryptic- |
|---|---|
| Metrics | Acceptor Donor |
| True Positive (TP) | 17,014 |
| True Negative (TN) | 12 |
| False Positive (FP) | 1355 |
| False Negative (FN) | 40 |
| Accuracy | 99.72% |
| Precision | 98.53% |
| Recall | 99.44% |
| F1-Score | 98.98% |
| Mathew's Correlation | 0.98 |
| Coefficient (MCC) Score | |

### Step 7. Cross-Validation of the Model

To estimate the ability of the machine learning (ML) model to generalize to non-training data, cross-validation was performed. The cross-validation was done using the K-Fold method with 10 folds (n_splits=10) and the Stratified K-Fold method with 3 folds (n_splits=3) to ensure that each fold was a representative sample of the whole dataset. Additionally, repeated random subsampling validation was performed using Shuffle Split cross-validation, which randomly splits the dataset into training and validation sets for each iteration. This was done using the ShuffleSplit method with 10 re-shuffling and splitting iterations (n_splits=10). Results are shown for cross-validation in Table 2.

TABLE 2

| Cross Validation Methods | Accuracy |
|---|---|
| K-Fold CV | 99.87% |
| Stratified K-Fold CV | 99.85% |
| Shuffle Split CV | 99.84% |

### Step 8: Validation of the Trained RF Model on a New External Dataset

Model validation is a critical component of building a supervised model and is essential for achieving good generalization performance. A sensible data-splitting strategy is crucial for model validation. To validate the created Random Forest (RF) model, the ClinVar and SCM datasets were used. The metrics for validation were observed and studied using the scikit-learn module once the pathogenicity was predicted for this data. The pathogenic and benign variants of 9549 real acceptors and real donors (pathogenic: 9,464, benign: 85) were obtained from ClinVar, and only SNPs were considered as the model was trained for SNP. To validate the pathogenicity model for cryptic acceptor and cryptic donor, 3638 (pathogenic: 3,619, benign: 19) variants were curated from the splice-site-creating mutations (SCM) dataset. The pathogenicity calls of these variants were re-classified based on the matching pathogenicity of the SQUIRL and S&S algorithms. Table 3 illustrates validation values.

TABLE 3

| | Clinvar Real & | SCM Dataset |
|---|---|---|
| | Cryptic- | Real & Cryptic- |
| | Acceptor/Donor | Acceptor/Donor |
| Metrics | (9549 Variants) | (3638 variants) |
| True Positive (TP) | 9463 | 3614 |
| True Negative (TN) | 81 | 10 |
| False Positive (FP) | 4 | 9 |
| False Negative (FN) | 1 | 5 |
| Accuracy | 99.14% | 99.58% |
| Precision | 52.35% | 73.62% |
| Recall | 89.57% | 82.00% |
| F1-Score | 54.22% | 77.16% |
| Mathew's Correlation | 0.193 | 0.55 |
| Coefficient (MCC) | | |
| Score | | |

### Step 9 : A Deep Learning Model to Predict the Pathogenicity in RSE Elements

### A. Data Collection

The same dataset of 61,401 RSE variants curated in step 1 will be used for building the deep learning model.

### B. Data Pre-Processing

Before training the deep learning model, the above dataset needs to be cleaned to remove any duplicates, missing values, and irrelevant data. The data is then processed and transformed into a compatible format for use in a deep learning model. The variant nucleotides of regulatory and splice elements are converted into numerical representations using one-hot encoding. Resulting data will be split into training and testing sets with a ratio of 80:20.

### C. Building the Model

A deep neural network (DNN) was used to predict the pathogenicity of mutations in regulatory and splice elements. The DNN model was designed by specifying architecture elements, including the number of hidden layers, the number of neurons in each layer, the activation functions, etc. The DNN will have multiple hidden layers, and each layer will contain multiple neurons. The output layer of the DNN has a single neuron that is responsible for making the final prediction of the pathogenicity of the mutation. This single neuron provides the final prediction based on the information received from the hidden layers and processed through the weights and biases of the neurons.

The model is then compiled by defining the optimizer, loss function, and metrics used for evaluation. The optimizer determines how the model will be updated during training, the loss function measures the accuracy of the predictions, and the metrics evaluate the model's performance.

### D. Training the Model

The deep learning model will be trained on the pre-processed data. During the training process, the model will learn to predict the pathogenicity of mutations in regulatory and splice elements. The model is then trained using the training data by running a number of iterations, also known as epochs. During each iteration, the model is updated based on the loss function and optimizer, and the metrics are evaluated to monitor the model's performance. We will use a loss function to measure the accuracy of the model and adjust the weights of the neurons in each layer to improve the model's performance.

### E. Evaluating the Model

Once the model is trained, the model was then evaluated on a test dataset. The evaluation was performed using metrics such as accuracy, precision, recall, and F1 score. If necessary, the model can be fine-tuned by adjusting the hyperparameters such as the number of hidden layers, the number of neurons in each layer, the activation functions, etc.

The above steps and nuances are used in their ML applications of the genome features described in the different embodiments in order to predict the different genomic features, and to identify the variants that are causal of disease and drug response phenotypes from among the millions of variants possible in the genome sequence.

### Computer System

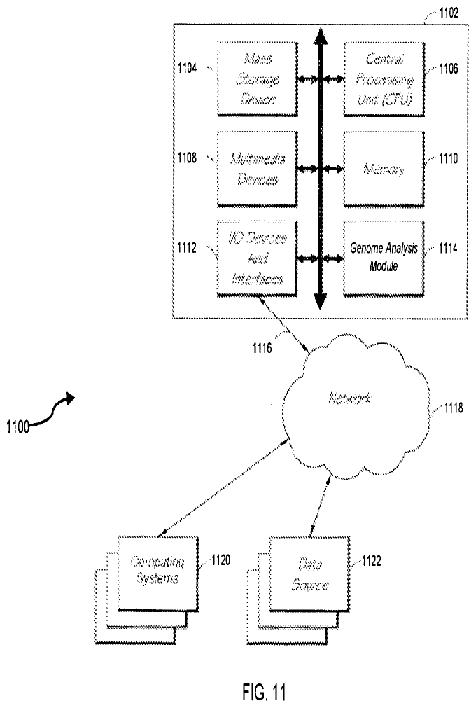

In some embodiments, the systems, processes, and methods described herein are implemented using a computing system, such as the one illustrated in FIG. 11. The example computer system 1102 is in communication with one or more computing systems 1120 and/or one or more data sources 1122 via one or more networks 1118. While FIG. 11 illustrates an embodiment of a computing system 1102, it is recognized that the functionality provided for in the components and modules of computer system 1102 can be combined into fewer components and modules, or further separated into additional components and modules.

The computer system 1102 can comprise a genome analysis module 1114 that carries out the functions, methods, acts, and/or processes described herein. The genome analysis module 1114 is executed on the computer system 1102 by a central processing unit 1106 discussed further below.

In general the word “module,” as used herein, refers to logic embodied in hardware or firmware or to a collection of software instructions, having entry and exit points . Modules are written in a program language, such as JAVA, C, or C++, or the like. Software modules can be compiled or linked into an executable program, installed in a dynamic link library, or can be written in an interpreted language such as BASIC, PERL, LAU, PHP or Python and any such languages. Software modules can be called from other modules or from themselves, and/or can be invoked in response to detected events or interruptions. Modules implemented in hardware include connected logic units such as gates and flip-flops, and/or can include programmable units, such as programmable gate arrays or processors.

Generally, the modules described herein refer to logical modules that can be combined with other modules or divided into sub-modules despite their physical organization or storage. The modules are executed by one or more computing systems, and can be stored on or within any suitable computer readable medium, or implemented in-whole or in-part within special designed hardware or firmware. Not all calculations, analysis, and/or optimization require the use of computer systems, though any of the above-described methods, calculations, processes, or analyses can be facilitated through the use of computers. Further, in some embodiments, process blocks described herein can be altered, rearranged, combined, and/or omitted.

### Computing System Components

The computer system 1102 includes one or more processing units (CPU) 1106, which can comprise a microprocessor. The computer system 1102 further includes a physical memory 1110, such as random access memory (RAM) for temporary storage of information, a read only memory (ROM) for permanent storage of information, and a mass storage device 1104, such as a backing store, hard drive, rotating magnetic disks, solid state disks (SSD), flash memory, phase-change memory (PCM), 3D XPoint memory, diskette, or optical media storage device. Alternatively, the mass storage device can be implemented in an array of servers. Typically, the components of the computer system 1102 are connected to the computer using a standards based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1102 includes one or more input/output (I/O) devices and interfaces 1112, such as a keyboard, mouse, touch pad, and printer. The I/O devices and interfaces 1112 can include one or more display devices, such as a monitor, that allows the visual presentation of data to a user. More particularly, a display device provides for the presentation of GUIs as application software data, and multi-media presentations, for example. The I/O devices and interfaces 1112 can also provide a communications interface to various external devices. The computer system 1102 can comprise one or more multi-media devices 1108, such as speakers, video cards, graphics accelerators, and microphones, for example.

### Computing System Device/Operating System

The computer system 1102 can run on a variety of computing devices, such as a server, a Windows server, a Structure Query Language server, a Unix Server, a personal computer, a laptop computer, and so forth. In other embodiments, the computer system 1102 can run on a cluster computer system, a mainframe computer system and/or other computing system suitable for controlling and/or communicating with large databases, performing high volume transaction processing, and generating reports from large databases. The computing system 1102 is generally controlled and coordinated by an operating system software, such as z/OS, Windows, Linux, UNIX, BSD, PHP, SunOS, Solaris, MacOS, ICloud services or other compatible operating systems, including proprietary operating systems. Operating systems control and schedule computer processes for execution, perform memory management, provide file system, networking, and I/O services, and provide a user interface, such as a graphical user interface (GUI), among other things.

### Network

The computer system 1102 illustrated in FIG. 11 is coupled to a network 1118, such as a LAN, WAN, or the Internet via a communication link 1116 (wired, wireless, or a combination thereof). Network 1118 communicates with various computing devices and/or other electronic devices. Network 1118 is communicating with one or more computing systems 1120 and one or more data sources 222. The genome analysis module 1114 can access or can be accessed by computing systems 1120 and/or data sources 1122 through a web-enabled user access point. Connections can be a direct physical connection, a virtual connection, and other connection type. The web-enabled user access point can comprise a browser module that uses text, graphics, audio, video, and other media to present data and to allow interaction with data via the network 1118.

The output module can be implemented as a combination of an all-points addressable display such as a cathode ray tube (CRT), a liquid crystal display (LCD), a plasma display, or other types and/or combinations of displays. The output module can be implemented to communicate with input devices 1112 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module can communicate with a set of input and output devices to receive signals from the user.

### Other Systems

The computing system 1102 can include one or more internal and/or external data sources (for example, data sources 1122). In some embodiments, one or more of the data repositories and the data sources described above can be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 1102 can also access one or more databases 1122. The databases 1122 can be stored in a database or data repository. The computer system 1102 can access the one or more databases 1122 through a network 1118 or can directly access the database or data repository through I/O devices and interfaces 1112. The data repository storing the one or more databases 1122 can reside within the computer system 1102.

### URLs and Cookies

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Doman Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

### Other Embodiments

While operations may be depicted in the drawings or described in the specification in a particular order, such operations need not be performed in the particular order shown or in sequential order, or that all operations be performed, to achieve desirable results. In particular, elements presented relating to GUI elements or displays to a user may be presented in any particular order to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the features for the system described herein can be provided separately, or integrated together (e.g., packaged together, or attached together).

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

### Operative Embodiments

In some aspects, the techniques described herein relate to a method of analysis of features, mutations, and genomes, the method including: receiving a plurality of nucleotides including a genetic element in a gene, wherein the plurality of nucleotides are assigned a position; calculating the frequency of mutations for each position within the genetic element, wherein the nucleotide at the position within the genetic element is replaced by an alternative nucleotide; calculating the total number of mutations for the sequence length of the genetic element; and calculating a deleteriousness score for each position based on the frequency of mutations.

In some aspects, the techniques described herein relate to a method, further including calculating a disease causality score, wherein the disease causality score is calculated based on the frequency of specific mutations divided by the total number of mutations calculated.

In some aspects, the techniques described herein relate to a method for comparing similarity between genetic features, the method including receiving a nucleotide sequence from a reference genome including at least one genetic element, wherein the at least one genetic element is selected from a list including: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof; identifying a first exon from the nucleotide sequence, wherein the first exon begins with an initiator codon, wherein the first exon ends with a first donor sequence; identifying a middle exon from the nucleotide sequence; identifying a last exon from the nucleotide sequence; and, annotating splicing and regulatory elements based on similarity scores or position weight matrix scores.

In some aspects, the techniques described herein relate to a method, wherein the similarity scores are computed from one of: determining the similarity score of an element by executing instructions from an algorithm selected from a group consisting of algorithms such as Shapiro-Senapathy algorithm, MaxEntScan algorithm, and NNSplice algorithm, stored in a memory; determining the similarity score of an element by executing instructions from a modified algorithm selected from a group consisting of algorithms such as Shapiro-Senapathy algorithm, MaxEntScan algorithm, and NNSplice algorithm, stored in a memory, based on the characteristics of a genetic element sequence signal such as length or variability; or determining a combined score of the group of algorithms based on an average or differentially weighted scores.

In some aspects, the techniques described herein relate to a computer implemented method for interpreting a genome, including: receiving a nucleotide string including at least one genetic element, wherein the at least one genetic element includes: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof; generating, for each base in the nucleotide string, at least one alternative nucleotide, thereby generating at least one alternative nucleotide string, wherein for each base in the alternative nucleotide string, the nucleotide differs compared to the same position of the nucleotide string; calculating a similarity score for the at least one genetic element for the nucleotide string and all alternative nucleotide string(s); and, calculating molecular effects, wherein the molecular effects include one or more of: exon skipping, intron retention, cryptic exon creation, or partial exon deletion.

In some aspects, the techniques described herein relate to a method, wherein the similarity scores are computed from one of: determining the similarity score of an element by executing instructions from an algorithm selected from a group consisting of algorithms such as Shapiro-Senapathy algorithm, MaxEntScan algorithm, and NNSplice algorithm, stored in a memory; determining the similarity score of an element by executing instructions from a modified algorithm selected from a group consisting of algorithms such as Shapiro-Senapathy algorithm, MaxEntScan algorithm, and NNSplice algorithm, stored in a memory, based on the characteristics of a genetic element sequence signal such as length or variability; or determining a combined score of the group of algorithms based on an average or differentially weighted scores.

In some aspects, the techniques described herein relate to a computer implemented method, further including, determining the frequency of mutations that occur at one or more nucleotide positions within the sequence of one or more genetic elements in one or more genes, causal of one or more diseases or drug response phenotypes, from one or more publications;

In some aspects, the techniques described herein relate to a computer implemented method, further including, determining the frequency of mutations that occur at one or more nucleotide positions within the sequence of one or more types of genetic elements (e.g., the donor), in one or more genes in one or more diseases from one or more publications containing the data;

In some aspects, the techniques described herein relate to a computer implemented method, further including, statistical graphing and plotting the frequencies of mutations at one or more sequence positions within one or more elements in one or more genes for one or more diseases in different graphical and tabular representations;

In some aspects, the techniques described herein relate to a computer implemented method, further including, determining the frequency of mutations at one or more sequence positions within one or more genetic elements, reported in one or more publications; and, determining the pathogenically high, medium, or low variable positions within the genetic element, based on the frequencies, indicative of the level of pathogenicity or deleteriousness of the position;

In some aspects, the techniques described herein relate to a computer implemented method, further including, training an AI/ML system with the variation of mutational frequencies including the high, medium and low variable positions of one or more genetic elements in a gene, and predicting the level of pathogenicity of mutations by the AI/ML system;

In some aspects, the techniques described herein relate to a computer implemented method, further including, determining a scoring system for each genetic element that occurs in a gene based on the differential mutations at each of the different sequence positions within each genetic element;

In some aspects, the techniques described herein relate to a computer implemented method, further including, a genetic element representing a real element or a cryptic element that occurs in a gene;

In some aspects, the techniques described herein relate to a computer implemented method, further including, determining a deleteriousness or disease-causality of a mutation within a particular genetic element within a gene based on the frequency of the nucleotide change of a particular mutation to any of the other three nucleotides at that particular position, relative to the change of nucleotides at all of the sequence positions within the element;

In some aspects, the techniques described herein relate to identifying disease-causing cryptic sites for a genetic element based on their locations in which disease-causing mutations occur at a high frequency from published data, and applying the disease-causality scoring algorithms to the cryptic element;

In some aspects, the techniques described herein relate to determining the disease-causality of a mutation in a cryptic site of an element from an individual, based on the disease-causality algorithm.

In some aspects, the techniques described herein relate to a method of analysis of features, mutations, genes, and genomes, the method including: receiving a plurality of nucleotides including a genetic element in a gene, wherein the plurality of nucleotides are assigned a position; calculating a frequency of mutations for each position within the genetic element based on publications, wherein the nucleotide at the position within the genetic element is replaced by an alternative nucleotide; calculating the total number of mutations for the sequence length of the genetic element; and calculating a deleteriousness score for each specific position based on the frequency of mutations at that position relative to the total number of mutations.

In some aspects, the techniques described herein relate to a method, further including calculating a disease causality score for each specific base change into any one of three other bases at every position in the element, wherein the disease causality score is calculated based on the frequency of specific base change divided by the total number of mutations at that position.

In some aspects, the techniques described herein relate to a method, further including: determining the frequency of mutations that occur at each nucleotide position within the sequence of the genetic element, wherein the mutations are retrieved from one or more publications.

In some aspects, the techniques described herein relate to a method, further including: statistical graphing and plotting the frequencies of mutations at one or more sequence positions within the genetic element, wherein statistically graphing and plotting are performed in different graphical and tabular representations

In some aspects, the techniques described herein relate to a method, further including determining the frequency of mutations at one or more sequence positions within the genetic element, reported in one or more publications; and, determining the high, medium, or low variable positions within the genetic element, based on the frequencies, indicative of the level of pathogenicity or deleteriousness of the position.

In some aspects, the techniques described herein relate to a method, wherein determining the high, medium or low variable positions includes training an AI/ML system with the frequency of mutations of one or more genetic elements in a gene.

In some aspects, the techniques described herein relate to a method, further including identifying disease-causing cryptic sites for a genetic element based on predetermined locations in which disease-causing mutations occur at a high frequency from published data; and, applying a disease-causality scoring algorithm to the cryptic element.

In some aspects, the techniques described herein relate to a method for identifying a gene in a raw DNA sequence, the method including receiving a nucleotide sequence from a reference genome, the reference genome including at least one genetic element, wherein the at least one genetic element is selected from a list including: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof; identifying a first exon from the nucleotide sequence, wherein the first exon begins with an initiator codon, wherein the first exon ends with a first donor sequence, and the first exon is bounded by an open reading frame (ORF); identifying one or more middle exons from the nucleotide sequence, wherein the middle exon starts with a first acceptor sequence and ends with a second donor sequence, and the middle exon is bounded by the open reading frame (ORF); identifying a last exon from the nucleotide sequence, wherein the last exons starts with a second acceptor sequence and ends with a stop codon, and the last exon is bounded by the open reading frame (ORF); and, annotating the splicing and regulatory elements within the gene based on similarity scores or position weight matrix scores.

In some aspects, the techniques described herein relate to a method, wherein the similarity scores are computed by: determining the similarity score of an element by executing instructions from an algorithm selected from a group consisting of: Shapiro-Senapathy algorithm, MaxEntScan algorithm, and NNSplice algorithm, stored in a memory; determining the similarity score of an element by executing instructions from a modified algorithm selected from a group consisting of: Shapiro-Senapathy algorithm, MaxEntScan algorithm, and NNSplice algorithm, stored in a memory; or determining a combined average or differentially weighted score based on a group of algorithms, wherein the group of algorithms consist of: Shapiro-Senapathy algorithm, MaxEntScan algorithm, and NNSplice algorithm, stored in a memory, or modifications thereof.

In some aspects, the techniques described herein relate to a method, wherein the genetic elements, or exons are identified based on a threshold of similarity scores.

In some aspects, the techniques described herein relate to a computer implemented method, further including, identifying the first exon, one or more middle exons, and the last exon of the gene, wherein the first exon, one or more middle exons, and the last exons are characterized by the highest similarity scores, wherein the similarity scores are calculated based on the at least one genetic elements within and surrounding the first exon, one or more middle exons, and the last exon; and, choosing the first exon, one or more middle exons, and the last exon of the gene based on the contiguity of the ORF of the consecutive exons starting from the first exon of a protein coding sequence, and the contiguity of protein domains over one or more exons, within the gene.

In some aspects, the techniques described herein relate to a computer implemented method, further including, determining a contiguous domain sequence of a protein domain, wherein the protein domain corresponds with the contiguous nucleotide sequence of either the first exon, the one or more middle exons, or the last exon; and, predicting that if a portion of the contiguous domain sequence is missing, then an exon is missing from the gene.

In some aspects, the techniques described herein relate to a computer implemented method, further including, determining the occurrence of one or more premature termination codons within a complete protein sequence, wherein the complete protein sequence is translated from the raw DNA sequence, wherein the premature termination codons indicate the presence of one or more cryptic exons; eliminating the one or more cryptic exons from a map of the gene; and, identifying the first exon, one or more middle exons, and the last exon of a complete gene without interfering stop codons.

In some aspects, the techniques described herein relate to a computer implemented method, including, receiving a nucleotide string including at least one genetic element, the at least one genetic element selected from: a 5′-UTR, a promoter, an enhancer, a silencer, an exon, an intron, a coding sequence, a non-protein coding RNA, a splice acceptor, a splice donor, a branch point site, a 3′-UTR, a Kozak sequence, a poly-A addition site or signal, or a cryptic version thereof, from a known protein coding gene, or a regulatory, splicing, or functional element of a non-protein coding RNA gene from a reference genome; generating one or more modified nucleotide strings, wherein each base on the one or more modified nucleotide strings is replaced compared to the nucleotide string, wherein replacing each base includes converting each base to a non-identical nucleotide; for the at least one genetic element, calculating the similarity score of the element for every of the one or more modified nucleotide strings; determining overall deleteriousness by comparing the similarity scores for the at least one genetic element for every one of the one or more modified nucleotide strings and for the nucleotide string; assigning a molecular effect, the molecular effect selected from one or more of: abolition, reduction or enhancement of transcription or translation, exon skipping, intron retention, cryptic exon creation or partial exon deletion of the deleterious mutation; and storing the information of the molecular effect for every one or more modified nucleotide strings in a memory.

In some aspects, the techniques described herein relate to a computer implemented method, further including, determining the molecular effect for every genetic element occurring throughout a genome, wherein the nucleotide string is part of the genome.

In some aspects, the techniques described herein relate to a computer implemented method, further including, receiving the nucleotide string including the at least one genetic element, wherein the nucleotide string is selected from a known protein coding gene, or wherein the nucleotide string is selected from a regulatory, splicing, or functional element of a non-protein coding RNA gene from a genome of an individual; identifying at least one variant in at the at least one genetic element, wherein identifying at least one variant is accomplished by comparing with the reference genome; comparing the molecular effect of mutations stored in the memory with the at least one variant, thereby generating at least one comparison; and storing a record of the at least one comparison in memory.

In some aspects, the techniques described herein relate to a computer implemented method, further including, determining the molecular effect for every at least one genetic element occurring throughout a genome, wherein the nucleotide string is part of the genome, wherein the genome is collected from the individual.

In some aspects, the techniques described herein relate to a computer implemented method, further including, determining the molecular effects of every genetic element for every gene occurring throughout the genome of an individual.

In some aspects, the techniques described herein relate to a computer implemented method, further including, evaluating the molecular effect of two or more variants for the at least one genetic element, wherein evaluation is assessed by comparing the similarity scores of the at least one genetic element against multiple variants of the at least one genetic element.

In some aspects, the techniques described herein relate to a computer implemented method, further including, assessing the molecular effect of two or more variants in two or more genetic elements, wherein the genetic elements are real or cryptic, wherein the genetic elements are located within an exon or intron, wherein assessing the molecular effect includes determining the similarity scores and other parameters of the two or more variants in two or more genetic elements.

In some embodiments, the system can be trained to recognize CRISPR recognition sequences in the genome. The training can include the CRISPR-Cas9 recognition sequences in graphical gene structure and sequence view. In addition, it can be trained to graphically illustrate the recognition sequences, their mutations and their aberrations in gene structure and sequence views.

A computer implemented method for automatically assessing genomic features comprising:
receiving an input dataset comprising one or more regulatory and one or more splicing elements in a gene set;
generating one or more similarity scores for the one or more regulatory and one or more splicing elements;
generating one or more pathogenic or strength altering mutations, wherein generating one or more pathogenic or strength altering mutations involves calculating pathogenicity of known mutations in the one or more regulatory and one or more splicing elements;
training an artificial intelligence program with the one or more regulatory and one or more splicing elements, wherein the one or more similarity scores are within a preset range;
generating an output dataset of splicing or regulatory elements, wherein the output dataset comprises a new set of genes; and
generating pathogenic or strength altering mutations for the new set of genes.

The computer implemented method of the above embodiments, further comprising,
receiving a plurality of nucleotides from one or more individuals with at least one genetic element, exon, intron or a gene;
identifying pathogenic or strength altering mutations in the plurality of nucleotides from one or more individuals.

The computer implemented method of the above embodiments, wherein generating pathogenic or strength altering mutations for the new set of genes identifies genetic elements causing phenotypes such as disease and drug response, including therapeutics and harmful side effects.

The computer implemented method of the above embodiments, further comprising,
training an artificial intelligence program with one or more known cryptic elements from the input dataset, wherein the one or more known cryptic elements include genetic environment of other genetic elements; and
generating as an output one or more novel cryptic element mutations causing disease, drug response and harmful side effects.

The computer implemented method of the above embodiments, further comprising,
identifying true and cryptic genetic elements in the new set of genes using a machine learning model, wherein the machine learning model is trained with one or more known true and cryptic genetic elements from the input dataset, wherein the one or more known true and cryptic genetic elements are categorized based on calculated similarity scores.

The computer implemented method of the above embodiments, wherein the input dataset is collected from one or more gene databases.
A computer implemented method of the above embodiments, further comprising,
sorting pathogenic or strength altering mutations from benign mutations.

A computer implemented method of the above embodiments, further comprising,
predicting deleterious or strength altering mutations in different genetic elements of the new set of genes.
A system configured for assessing genomic features, comprising a system configured to carry out the method of the above embodiments.

What is claimed is:

1. A computer implemented method for automatically assessing genomic features comprising:
receiving an input dataset comprising one or more regulatory and/or one or more splicing elements in a gene set;
generating one or more similarity scores for the one or more regulatory and/or one or more splicing elements;
generating one or more pathogenic or strength altering mutations, wherein generating one or more pathogenic or strength altering mutations involves calculating pathogenicity of known mutations in the one or more regulatory and/or one or more splicing elements, and the difference between the scores before and after mutation;
training an artificial intelligence program with the one or more regulatory and/or one or more splicing elements, wherein the one or more similarity scores are within a preset range;
training the artificial intelligence program with known pathogenic or strength altering mutations in splicing or regulatory elements in a set of genes with known splicing and regulatory elements, genomic positions, and similarity scores;
generating an output dataset of splicing or regulatory elements, wherein the input dataset comprises a new set of genes; and
generating pathogenic or strength altering mutations for the new set of genes.

2. The computer implemented method of claim 1, further comprising,
receiving a plurality of nucleotides from one or more individuals with at least one genetic element, exon, intron or a gene;
identifying pathogenic or strength altering mutations in the plurality of nucleotides from one or more individuals based on the trained artificial intelligence program.

3. The computer implemented method of claim 1, further comprising,
receiving a plurality of nucleotides from one or more individuals with at least one genetic element, exon, intron or a gene;
identifying one or more molecular effects due to pathogenic or strength altering mutations in the plurality of nucleotides from one or more individuals based on the trained artificial intelligence program.

4. The computer implemented method of claim 1, wherein generating pathogenic or strength altering mutations in genetic elements for the new set of genes identifies phenotypes such as disease and drug response, including therapeutics and harmful side effects.

5. The computer implemented method of claim 1, further comprising,
training an artificial intelligence program with one or more known cryptic elements from the input dataset, wherein the one or more known cryptic elements include genetic environment of other genetic elements; and their pathogenic or strength altering mutations causing various phenotypes in a set of known genes;
generating as an output one or more novel cryptic element mutations causing disease, drug response and harmful side effects.

6. The computer implemented method of claim 1, further comprising,
identifying true and cryptic genetic elements in the new set of genes using a machine learning model, wherein the machine learning model is trained with one or more known true and cryptic genetic elements from the input dataset, wherein the one or more known true and cryptic genetic elements are categorized based on calculated similarity scores and genomic positions in known genes.

7. A computer implemented method of claim 1, further comprising,
the AI model sorting pathogenic or strength altering mutations from benign mutations.

8. The computer implemented method of claim 1, further comprising,
identifying pathogenic or strength altering mutations in the new set of genes using a machine learning model, wherein the machine learning model is trained with known pathogenic and strength altering mutations and non-deleterious or benign mutations, wherein the pathogenic and strength altering mutations and non-deleterious or benign mutations are categorized based on calculated similarity scores, genomic positions in known genes, and their genetic environment of other elements and their parameters within the genes and the genome.

9. A computer implemented method of claim 1, further comprising,
the trained AI model predicting deleterious or strength altering mutations in different genetic elements of the new set of genes.

10. A system configured for assessing genomic features, comprising a system configured to carry out the method of claim 1.
